(19) United States
(12) Patent Application Publication
(10) Pub. No.: US 2018/0277013 A1
(43) Pub. Date: Sep. 27, 2018

(54) MESSAGING SYSTEM

(71) Applicant: KONINKLIJKE PHILIPS N.V., Eindhoven (NL)

(72) Inventors: AART TIJMEN VAN HALTEREN, GELDROP (NL); JULIENKA MOLLEE, EINDHOVEN (NL); MICHEL KLEIN, EINDHOVEN (NL); ADNAN MANZOOR, EINDHOVEN (NL); ANOUK MIDDELWEERD, EINDHOVEN (NL); SASKIA TE VELDE, EINDHOVEN (NL)

(21) Appl. No.: 15/937,166

(22) Filed: Mar. 27, 2018

Related U.S. Application Data
(60) Provisional application No. 62/486,609, filed on Apr. 18, 2017.
(60) Provisional application No. 62/477,034, filed on Mar. 27, 2017.

(51) Int. Cl. G09B 19/00; G16H 20/30; A63B 24/00; A63B 71/06; G09B 5/02; H04W 4/12

(52) U.S. Cl. CPC G09B 19/00; A63B 2220/75; A63B 24/0062; A63B 71/06; G09B 5/02; H04W 4/12; A63B 2220/12; A63B 2225/50; A63B 2225/54; A63B 2220/803; A63B 2220/836; A63B 2230/06; A63B 2230/30; A63B 2230/42; A63B 2230/65; A63B 2230/60; A63B 2230/50; A63B 2220/72; A63B 2220/74; G16H 20/30

(57) ABSTRACT

In an embodiment, an apparatus is presented that assesses an awareness of a user about a need for influencing a change in activity levels of the user by receiving information about activity levels of a user and receiving user input about user activity, associates the user with a category among plural categories based on the awareness assessment, and provides messaging to the user based on simulated effects of targeting various personal determinants to achieve a strong influence on the user's behavior.

### CROSS-REFERENCE TO RELATED APPLICATIONS

This patent application claims the priority benefit under 35 U.S.C. § 119(e) of U.S. Provisional Application No. 62/477,034 filed on Mar. 27, 2017, and U.S. Provisional Application No. 62/486,609 filed on Apr. 18, 2017 the contents of which are herein incorporated by reference.

### FIELD OF THE INVENTION

The present invention is generally related to health and wellness monitoring and, more particularly, messaging to improve health and wellness.

### BACKGROUND OF THE INVENTION

Physical inactivity is an increasingly serious health problem. The World Health Organization (WHO) has identified that physical inactivity is the fourth leading risk factor for global mortality, and estimates that a lack of physical activity leads to 3.2 million deaths globally. Physical inactivity owes much to modern sedentary lifestyles, which according to WHO are led by 60% to 85% of people in the world. One aspect of sedentary lifestyles is that people are more inclined to use passive modes of transportation. Active travelling modes, including biking and walking, can contribute to a healthy level of physical activity. Another aspect of sedentary lifestyle is related to the work environment, where much work is done by people seated in chairs in front of computers. Research suggests that having desk jobs increases health risks up to 50%. Integrating small activities in work routines can help to increase physical activity and lower health risks.

Stimulating people to have a healthy physical activity level is a big challenge. It is known that personalized instruction (e.g., coaching, personal training, etc.) is more effective than a one-size-fits-all approach. One reason is that such personalized instruction is typically more persuasive to people. The concerns specific to the person are more likely to be addressed by personalized instruction, and thus, the person is more likely to take steps toward increasing physical activity. Yet, personalized instruction by a human can be an expensive solution that is out of reach for individuals with few resources, and interaction with a human coach is typically limited to particular sessions when both the human coach and coachee have availability for interaction. Additionally, a human coach can only interact with a limited number of coaches per day. On a large scale, providing a human coach for every person or insured patient in need of lifestyle changes would be extremely expensive for an insurance company and society. Yet, for example, with the global epidemic of diabetes and heart disease, there is a great need for coaching to help people change their lifestyles to reduce their mortality risks. Automated systems exist for personalized training, but such systems either fail to provide for messaging or provide only generalized messaging. Improvements in helping people make lifestyle changes could have profound impacts on the quality of life for people and reduce the high costs of medical care related to chronic diseases by preventing such diseases.

### SUMMARY OF THE INVENTION

One object of the present invention is to determine factors for which improvement may lead to the strongest improvement in behavior. Another object is to select a message that exploits these factors in more substantial ways than a generalized messaging approach. To better address such concerns, in a first aspect of the invention, an apparatus is presented that assesses an awareness of a user about a need for influencing a change in activity levels of the user by receiving information about activity levels of a user and receiving user input about user activity, associates the user with a category among plural categories based on the awareness assessment, and provides messaging to the user based on simulated effects of targeting various personal determinants to achieve a strong influence on the user's behavior. The invention addresses a problem in the art of over-generalized messaging (or a complete lack of messaging) that has less influence on changing the activity patterns of a user compared to more personalized messaging, as well as mitigating the computational complexity of processing an extensive set of rules preliminary to each message to determine aspects of the user to personalize, since computations (e.g., simulations) are not run every time a message is provided but rather on a less frequent basis (e.g., weekly and stored).

In one embodiment, the apparatus determines which category to associate with the user by evaluating rules that associate an objective binary measure of the physical activity and a subjective binary measure of the user input to each of a plurality of categories. The use of a streamlined set of rules to ascertain activity patterns or behavior serves to focus messaging in a way that is personal to a user.

In one embodiment, the apparatus estimates an effect of improving each personal determinant by running simulations through a computational model. By running the simulations, messages that may yield the most promising effect on user behavior can be targeted.

In one embodiment, the apparatus runs the simulations by: estimating a current state of each of the personal determinants; inputting values corresponding to the current state; increasing a value for respective one or more targeted personal determinants according to a hypothesized effect of sending a message about the one or more targeted personal determinants; based on the increased value or values, estimating an effect on behavior; and providing an ordered list of personal determinants and a corresponding probability, the ordering based on a most promising effect on the corresponding behavior. By estimating the state, the apparatus can determine how strongly the personal determinant is present (e.g., how strong are the user's intentions, how high is the user's sense of self-efficacy, etc.). The personal determinants are represented as concepts in the computational model (e.g., with numerical values ranging between 0 and 1), as is the behavior of the user, with one aim to improve the behavior based on the values of the behavior in the simulations. That is, one outcome of the simulations is to provide a predicted behavior value for each of the hypothesized effects of targeting one of the personalized determinants, enabling a determination of the best personal determinant to influence behavior.

These and other aspects of the invention will be apparent from and elucidated with reference to the embodiment(s) described hereinafter.

### BRIEF DESCRIPTION OF THE DRAWINGS

Many aspects of the invention can be better understood with reference to the following drawings, which are diagrammatic. The components in the drawings are not necessarily to scale, emphasis instead being placed upon clearly illustrating the principles of the present invention. Moreover, in the drawings, like reference numerals designate corresponding parts throughout the several views.

FIG. 1 is a schematic diagram that illustrates an example environment in which a messaging system is used, in accordance with an embodiment of the invention.

FIG. 2 is a schematic diagram that illustrates an example wearable device in which all or a portion of the functionality of a messaging system may be implemented, in accordance with an embodiment of the invention.

FIG. 3 is a schematic diagram that illustrates an example electronics device in which all or a portion of the functionality of a messaging system may be implemented, in accordance with an embodiment of the invention.

FIG. 4 is a schematic diagram that illustrates an example computing device in which all or a portion of the functionality of a messaging system may be implemented, in accordance with an embodiment of the invention.

FIG. 5 is a flow diagram that illustrates an example process implemented by a messaging system, in accordance with an embodiment of the invention.

FIG. 6 is a schematic diagram that illustrates example rules used by a messaging system in a user awareness ascertainment phase of the messaging system, in accordance with an embodiment of the invention.

FIG. 7 is a flow diagram that illustrates an example process implemented by a messaging system in suggesting a domain selection/recommendation and prompting a user to select the domain selection/recommendation, in accordance with an embodiment of the invention.

FIG. 8 is a schematic diagram that illustrates an example data structure of messages and associated meta information as used in a messaging system, in accordance with an embodiment of the invention.

FIG. 9 is a schematic diagram that illustrates a graphical representation of a computational model used by the messaging system, in accordance with an embodiment of the invention.

FIG. 10 is a screen diagram that illustrates an example user interface used by the messaging system in providing messaging to a user, in accordance with an embodiment of the invention.

FIG. 11 is a flow diagram that illustrates an example messaging process, in accordance with an embodiment of the invention.

### DETAILED DESCRIPTION OF EMBODIMENTS

Disclosed herein are certain embodiments of a messaging system, apparatus, and method (herein, also collectively referred to as a messaging system) that interprets activity patterns or behavior and chooses the most likely personal determinant to effectuate tailoring and personalization of health and/or wellness instruction (e.g., coaching, including healthy lifestyle coaching). In general, the messaging system effectuates tailoring and personalization of healthy lifestyle coaching using a reasoning engine that assesses a user's awareness of a need for influencing a change in activity levels of the user, determines a category of influence or type of messaging (e.g., coaching, feedback, education) based on the awareness assessment, suggests a coaching domain based on selection of the coaching category and solicits a goal, finds the most promising personal determinants to effectuate that goal, and selects and tailors relevant messages based at least on the personal determinants targeted for messaging and further based on other (contextual) information to filter out irrelevant messages, including filtering based on the day of the week and/or time of day the messaging is relevant, whether a goal was achieved or not during a previous time period, whether the user is on track to achieve a current goal, forecasted weather conditions, etc. Personal determinants (also referred to herein as coaching determinants) comprise personal, psychological factors that influence one's (health) behavior. As explained further below, the personal determinants are used as concepts in a computational model that is used to run simulations and as possible targets for a coaching action.

Digressing briefly, current personalized training systems either neglect to provide for messaging, or provide over-generalized messaging in attempting to achieve behavioral change. Through the use of a limited set of rules to obtain user awareness and determine the type of category and domain to allot the user, and also through simulations performed to ascertain personal determinants to target, messages provided by certain embodiments of a messaging system can be tailored to each user in a personalized fashion to better influence behavioral or activity pattern change.

Having summarized certain features of a messaging system of the present disclosure, reference will now be made in detail to the description of a messaging system as illustrated in the drawings. While a messaging system will be described in connection with these drawings, there is no intent to limit messaging systems to the embodiment or embodiments disclosed herein. For instance, though described in the context of health management services, certain embodiments of a messaging system may be used to influence the behavior of a user in other contexts, including the areas of finance or other business or personnel management. Further, although the description identifies or describes specifics of one or more embodiments, such specifics are not necessarily part of every embodiment, nor are all various stated advantages necessarily associated with a single embodiment or all embodiments. On the contrary, the intent is to cover all alternatives, modifications and equivalents consistent with the disclosure as defined by the appended claims. Further, it should be appreciated in the context of the present disclosure that the claims are not necessarily limited to the particular embodiments set out in the description.

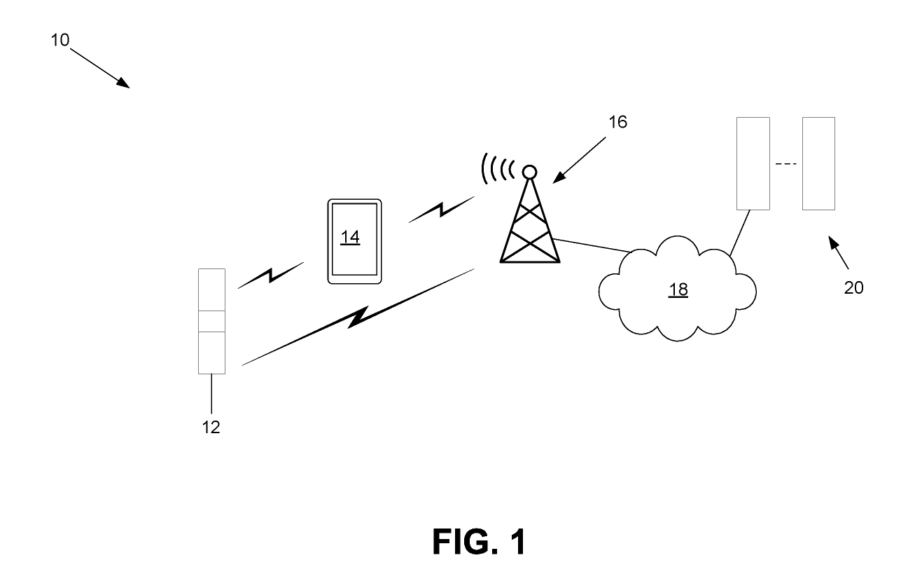

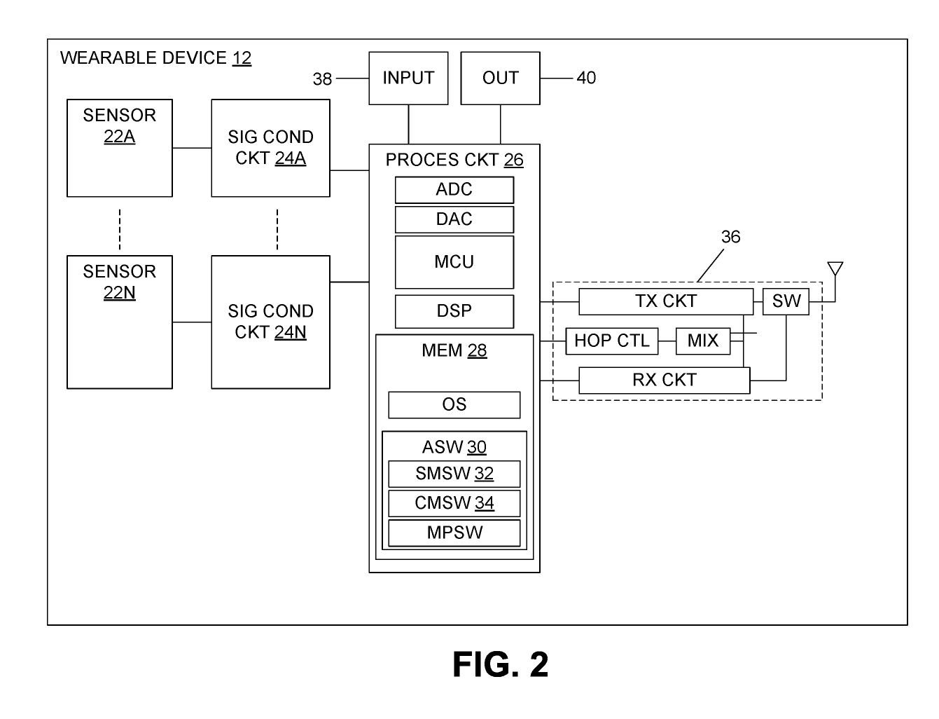

Referring now to FIG. 1, shown is an example environment 10 in which certain embodiments of a messaging system may be implemented. It should be appreciated by one having ordinary skill in the art in the context of the present disclosure that the environment 10 is one example among many, and that some embodiments of a messaging system may be used in environments with fewer, greater, and/or different components that those depicted in FIG. 1. The environment 10 comprises a plurality of devices that enable communication of information throughout one or more networks. The depicted environment 10 comprises a wearable device 12, an electronics (portable) device 14, a cellular network 16, a wide area network 18 (e.g., also described herein as the Internet), and a remote computing system 20. Note that the wearable device 12 and the electronics device 14 are also referred to as user devices. The wearable device 12, as described further in association with FIG. 2, is typically worn by the user (e.g., around the wrist or torso or attached to an article of clothing), and comprises a plurality of sensors that track physical activity of the user (e.g., steps, swim strokes, pedaling strokes, sports activities, etc.), sense/measure or derive physiological parameters (e.g., heart rate, respiration, skin temperature, etc.) based on the sensor data, and optionally sense various other parameters (e.g., outdoor temperature, humidity, location, etc.) pertaining to the surrounding environment of the wearable device 12. For instance, in some embodiments, the wearable device 12 may comprise a global navigation satellite system (GNSS) receiver, including a GPS receiver, which tracks and provides location coordinates (e.g., latitude, longitude, altitude) for the device 12. Other information associated with the recording of coordinates may include speed, accuracy, and a time stamp for each recorded location. In some embodiments, the location information may be in descriptive form, and geofencing (e.g., performed locally or external to the wearable device 12) is used to transform the descriptive information into coordinate numbers. In some embodiments, the wearable device 12 may comprise indoor location technology, including beacons, RFID or other coded light technologies, WiFi, etc. In some embodiments, GNSS functionality may be performed at the electronics device 14 in addition to, or in lieu of, such functionality being performed at the wearable device 12. Some embodiments of the wearable device 12 may include a motion or inertial tracking sensor, including an accelerometer and/or a gyroscope, providing movement data of the user (e.g., to detect limb movement and type of limb movement to facilitate the determination of whether the user is engaged in sports activities, stair walking, or bicycling). A representation of such gathered data may be communicated to the user via an integrated display on the wearable device 12 and/or on another device or devices.

Also, such data gathered by the wearable device 12 may be communicated (e.g., continually, periodically, and/or aperiodically, including upon request) to one or more electronics devices, such as the electronics device 14 or via the cellular network 16 to the computing system 20. Such communication may be achieved wirelessly (e.g., using near field communications (NFC) functionality, Blue-tooth functionality, 802.11-based technology, etc.) and/or according to a wired medium (e.g., universal serial bus (USB), etc.). Further discussion of the wearable device 12 is described below in association with FIG. 2.

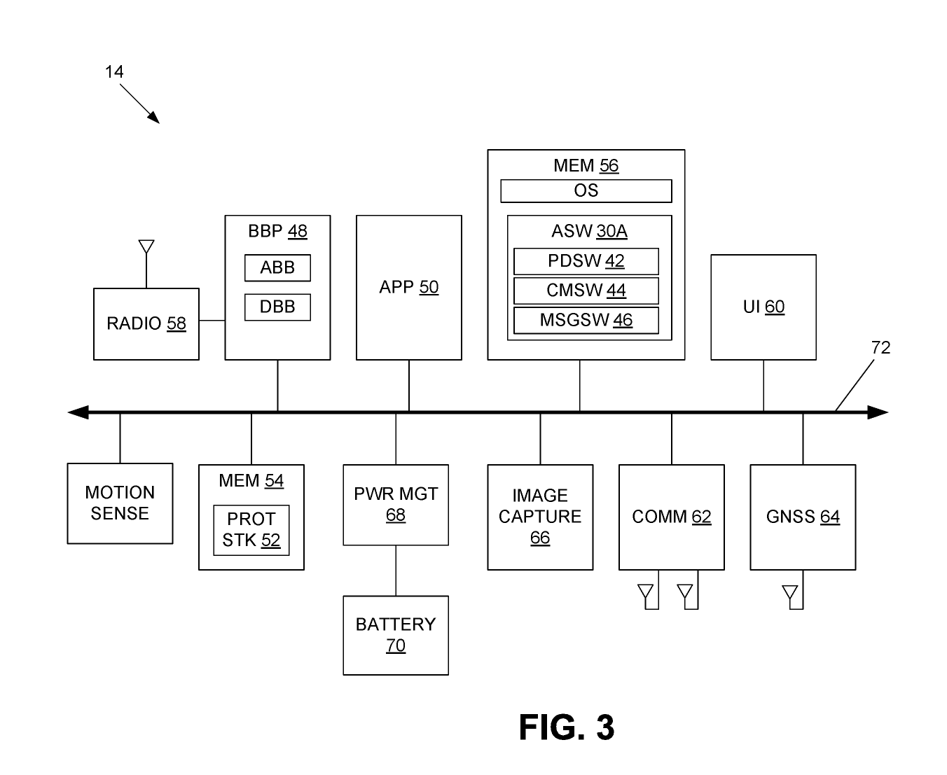

The electronics device 14 may be embodied as a smartphone, mobile phone, cellular phone, pager, stand-alone image capture device (e.g., camera), laptop, workstation, among other handheld and portable computing/communication devices, including communication devices having wireless communication capability, including telephony functionality. In the depicted embodiment of FIG. 1, the electronics device 14 is a smartphone, though it should be appreciated that the electronics device 14 may take the form of other types of devices as described above. Further discussion of the electronics device 14 is described below in association with FIG. 3, with smartphone and electronics device 14 used interchangeably hereinafter. In one embodiment, the electronics device 14 runs an application for the messaging system to facilitate communications with the wearable device 12, receive input from a user (e.g., via questionnaires), access location services (e.g., to monitor the user's location via GNSS functionality and/or user input), and access communications services (e.g., via web browser functionality) with a web service.

The cellular network 16 may include the necessary infrastructure to enable cellular communications by the electronics device 14 and optionally the wearable device 12. There are a number of different digital cellular technologies suitable for use in the cellular network 16, including: GSM, GPRS, CDMAOne, CDMA2000, Evolution-Data Optimized (EV-DO), EDGE, Universal Mobile Telecommunications System (UMTS), Digital Enhanced Cordless Telecommunications (DECT), Digital AMPS (IS-136/TDMA), and Integrated Digital Enhanced Network (iDEN), among others.

The wide area network 18 may comprise one or a plurality of networks that in whole or in part comprise the Internet. The electronics device 14 and optionally wearable device 12 access one or more of the devices of the computing system 20 via the Internet 18, which may be further enabled through access to one or more networks including PSTN (Public Switched Telephone Networks), POTS, Integrated Services Digital Network (ISDN), Ethernet, Fiber, DSL/ADSL, among others.

The computing system 20 comprises one or more devices coupled to the wide area network 18, including one or more computing devices networked together, including an application server(s) and data storage. The computing system 20 may serve as a cloud computing environment (or other server network) for the electronics device 14 and/or wearable device 12, performing processing and data storage on behalf of (or in some embodiments, in addition to) the electronics devices 14 and/or wearable device 12. When embodied as a cloud service or services, the device(s) of the remote computing system 20 may comprise an internal cloud, an external cloud, a private cloud, or a public cloud (e.g., commercial cloud). For instance, a private cloud may be implemented using a variety of cloud systems including, for example, Eucalyptus Systems, VMWare vSphere®, or Microsoft® HyperV. A public cloud may include, for example, Amazon EC2®, Amazon Web Services®, Terremark®, Savvis®, or GoGrid®. Cloud-computing resources provided by these clouds may include, for example, storage resources (e.g., Storage Area Network (SAN), Network File System (NFS), and Amazon S3®), network resources (e.g., firewall, load-balancer, and proxy server), internal private resources, external private resources, secure public resources, infrastructure-as-a-services (IaaSs), platform-as-a-services (PaaSs), or software-as-a-services (SaaSs). The cloud architecture of the devices of the remote computing system 20 may be embodied according to one of a plurality of different configurations. For instance, if configured according to MICROSOFT AZURE™, roles are provided, which are discrete scalable components built with managed code. Worker roles are for generalized development, and may perform background processing for a web role. Web roles provide a web server and listen for and respond to web requests via an HTTP (hypertext transfer protocol) or HTTPS (HTTP secure) endpoint. VM roles are instantiated according to tenant defined configurations (e.g., resources, guest operating system). Operating system and VM updates are managed by the cloud. A web role and a worker role run in a VM role, which is a virtual machine under the control of the tenant. Storage and SQL services are available to be used by the roles. As with other clouds, the hardware and software environment or platform, including scaling, load balancing, etc., are handled by the cloud.

In some embodiments, the devices of the remote computing system 20 may be configured into multiple, logically-grouped servers (run on server devices), referred to as a server farm. The devices of the remote computing system 20 may be geographically dispersed, administered as a single entity, or distributed among a plurality of server farms, executing one or more applications on behalf of one or more of the electronic devices 14 and/or wearable device 12. The devices of the remote computing system 20 within each farm may be heterogeneous. One or more of the devices may operate according to one type of operating system platform (e.g., WINDOWS NT, manufactured by Microsoft Corp. of Redmond, Wash.), while one or more of the other devices may operate according to another type of operating system platform (e.g., Unix or Linux). The group of devices of the remote computing system 20 may be logically grouped as a farm that may be interconnected using a wide-area network (WAN) connection or medium-area network (MAN) connection. The devices of the remote computing system 20 may each be referred to as a file server device, application server device, web server device, proxy server device, or gateway server device.

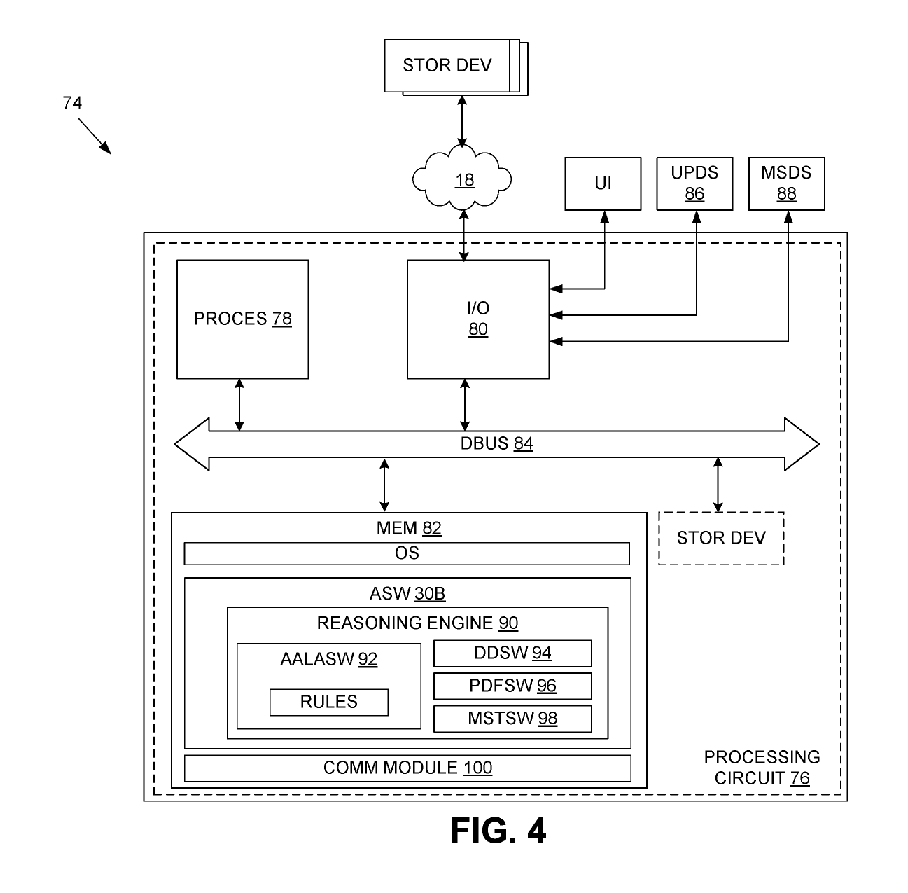

In one embodiment, the computing system 20 may comprise a web server that provides a web site that can be used by users to view their information (e.g., monitored activity, inputted information, such as entered via questionnaire, etc.). The computing system 20 receives data collected via one or more of the wearable device 12 or electronics device 14 and/or other devices or applications, stores the received data in a user profile data structure (e.g., database), processes the information to determine appropriate personal determinants to target, and delivers messaging to the electronics device 14 and/or wearable device 12. The computing system 20 is programmed to handle the operations of one or more health or wellness programs implemented on the wearable device 12 and/or electronics device 14 via the networks 16 and/or 18. For example, the computing system 20 processes user registration requests, user device activation requests, user information updating requests, data uploading requests, data synchronization requests, etc. The data received at the computing system 20 may be a plurality of measurements pertaining to the parameters, for example, body movements and activities, heart rate, respiration rate, blood pressure, body temperature, light and visual information, etc. and the corresponding context. Based on the data observed during a period of time for each user, the computing system 20 generates tailored messaging for delivery via the networks 16 and/or 18 for presentation on devices 12 and/or 14. In some embodiments, the computing system 20 is configured to be a backend server for a health-related program or a health-related application implemented on the mobile devices. The functions of the computing system 20 described above are for illustrative purpose only. The present disclosure is not intended to be limiting. The computing system 20 may be a general computing server device or a dedicated computing server device. The computing system 20 may be configured to provide backend support for a program developed by a specific manufacturer. However, the computing system 20 may also be configured to be interoperable across other server devices and generate information in a format that is compatible with other programs. In some embodiments, one or more of the functionality of the computing system 20 may be performed at the respective devices 12 and/or 14. Further discussion of the computing system 20 is described below in association with FIG. 4.

An embodiment of a messaging system may comprise the wearable device 12, the electronics device 14, and/or the computing system 20. In other words, one or more of the aforementioned devices 12, 14, and devices of the remote computing system 20 may implement the functionality of the messaging system. For instance, the wearable device 12 may comprise all of the functionality of a messaging system, enabling the user to avoid the need for Internet connectivity and/or carrying a smartphone 14 around. In some embodiments, the functionality of the messaging system may be implemented using a combination of the wearable device 12 and the electronics device 14 and/or the computing system 20 (with or without the electronics device 14). For instance, the wearable device 12 and/or the electronics device 14 may present messages via a user interface and provide sensing functionality, yet rely on remote data structures and/or processing of the remote computing systems 20.

As an example, the wearable device 12 may monitor activity of the user, and communicate sensed parameters (e.g., movement data, physiological data, etc.) to the electronics device 14. The electronics device 14 may ascertain the context of the data (e.g., the location) and receive additional input (e.g., responses to questionnaires, etc.) and communicate the wearable device data and electronics device-acquired data to a device of the remote computing system 20. The device or devices of the remote computing system 20 may use a reasoning engine to assess the user's current activity level and awareness, educate, provide feedback, and/or suggest a coaching domain and solicit a goal, find the most promising personal determinants via running of simulations, and select and tailor (customize) messages for provision to the user. The messaging may be provided to the user via a user interface (e.g., display screen, speaker) of the wearable device 12 and/or the electronics device 14 (or other device).

Attention is now directed to FIG. 2, which illustrates an example wearable device 12 in which all or a portion of the functionality of a messaging system may be implemented. That is, FIG. 2 illustrates an example architecture (e.g., hardware and software) for the example wearable device 12. It should be appreciated by one having ordinary skill in the art in the context of the present disclosure that the architecture of the wearable device 12 depicted in FIG. 2 is but one example, and that in some embodiments, additional, fewer, and/or different components may be used to achieve similar and/or additional functionality. In one embodiment, the wearable device 12 comprises a plurality of sensors 22 (e.g., 22A-22N), one or more signal conditioning circuits 24 (e.g., SIG COND CKT 24A-SIG COND CKT 24N) coupled respectively to the sensors 22, and a processing circuit 26 (PROCES CKT) that receives the conditioned signals from the signal conditioning circuits 24. In one embodiment, the processing circuit 26 comprises an analog-to-digital converter (ADC), a digital-to-analog converter (DAC), a microcontroller unit (MCU), a digital signal processor (DSP), and memory (MEM) 28. In some embodiments, the processing circuit 26 may comprise fewer or additional components than those depicted in FIG. 2. For instance, in one embodiment, the processing circuit 26 may consist of the microcontroller. In some embodiments, the processing circuit 26 may include the signal conditioning circuits 24. The memory 28 comprises an operating system (OS) and application software (ASW) 30. The application software 30 comprises a plurality of software modules (e.g., executable code/instructions) including sensor measurement software (SMSW) 32 and communications software (CMSW) 34. In some embodiments, the application software 30 may comprise message presentation software (MPSW) that presents messaging, including a dashboard of information. For purposes of illustration, the following description assumes the messaging to be provided via a user interface of the electronics device 14, with the understanding that similar functionality may be provided in some embodiments via the wearable device 12. Further, note that in some embodiments, the application software 30 may include additional software modules that receive user input and implement a reasoning engine as described herein as being performed at a device of the remote computing system 20 (FIG. 1). For purposes of brevity, the description about the application software 30 hereinafter is premised on the assumption that a device or devices of the remote computing system 20 comprise the reasoning engine.

The sensor measurement software 32 comprises executable code to process the signals (and associated data) measured by the sensors 22 and record and/or derive physiological parameters, such as heart rate, blood pressure, respiration, perspiration, etc. and movement and/or location data.

The communications software 34 comprises executable code/instructions to enable a communications circuit 36 of the wearable device 12 to operate according to one or more of a plurality of different communication technologies (e.g., NFC, Bluetooth, Wi-Fi, including 802.11, GSM, LTE, CDMA, WCDMA, Zigbee, etc.). The communications software 34 instructs and/or controls the communications circuit 36 to transmit the raw sensor data and/or the derived information from the sensor data to the computing system 20 (e.g., directly via the cellular network 16, or indirectly via the electronics device 14). The communications software 34 may also include browser software in some embodiments to enable Internet connectivity. The communications software 34 may also be used to access certain services, such as mapping/place location services, which may be used to determine a context for the sensor data. These services may be used in some embodiments of a messaging system, and in some instances, may not be used. In some embodiments, the location services may be performed by a client-server application running on the electronics device 14 and a device of the remote computing system 20.

As indicated above, in one embodiment, the processing circuit 26 is coupled to the communications circuit 36. The communications circuit 36 serves to enable wireless communications between the wearable device 12 and other devices, including the electronics device 14 and/or device(s) of the computing system 20, among other devices. The communications circuit 36 is depicted as a Bluetooth circuit, though not limited to this transceiver configuration. For instance, in some embodiments, the communications circuit 36 may be embodied as any one or a combination of an NFC circuit, Wi-Fi circuit, transceiver circuitry based on Zigbee, 802.11, GSM, LTE, CDMA, WCDMA, among others such as optical or ultrasonic based technologies. The processing circuit 26 is further coupled to input/output (I/O) devices or peripherals, including an input interface 38 (INPUT) and the output interface 40 (OUT). Note that in some embodiments, functionality for one or more of the aforementioned circuits and/or software may be combined into fewer components/modules, or in some embodiments, further distributed among additional components/modules or devices. For instance, the processing circuit 26 may be packaged as an integrated circuit that includes the microcontroller (microcontroller unit or MCU), the DSP, and memory 28, whereas the ADC and DAC may be packaged as a separate integrated circuit coupled to the processing circuit 26. In some embodiments, one or more of the functionality for the above-listed components may be combined, such as functionality of the DSP performed by the microcontroller.

The sensors 22 are selected to perform detection and measurement of a plurality of physiological and behavioral parameters (e.g., typical behavioral parameters or activities including walking, running, cycling, and/or other activities, including shopping, walking a dog, working in the garden, sports activities, etc.), including heart rate, heart rate variability, heart rate recovery, blood flow rate, activity level, muscle activity (e.g., movement of limbs, repetitive movement, core movement, body orientation/position, power, speed, acceleration, etc.), muscle tension, blood volume, blood pressure, blood oxygen saturation, respiratory rate, perspiration, skin temperature, body weight, and body composition (e.g., body mass index or BMI). At least one of the sensors 22 may be embodied as movement detecting sensors, including inertial sensors (e.g., gyroscopes, single or multi-axis accelerometers, such as those using piezoelectric, piezoresistive or capacitive technology in a microelectromechanical system (MEMS) infrastructure for sensing movement). In some embodiments, at least one of the sensors 22 may include GNSS sensors, including a GPS receiver to facilitate determinations of distance, speed, acceleration, location, altitude, etc. (e.g., location data, or generally, sensing movement), in addition to or in lieu of the accelerometer/gyroscope and/or indoor tracking (e.g., ibeacons, WiFi, coded-light based technology, etc.). In some embodiments, GNSS sensors may be included in the electronics device 14 in addition to, or in lieu of, those residing in the wearable device 12. The sensors 22 may also include flex and/or force sensors (e.g., using variable resistance), electromyographic sensors, electrocardiographic sensors (e.g., EKG, ECG), magnetic sensors, photoplethysmographic (PPG) sensors, bio-impedance sensors, infrared proximity sensors, acoustic/ultrasonic/audio sensors, a strain gauge, galvanic skin/sweat sensors, pH sensors, temperature sensors, pressure sensors, and photocells. The sensors 22 may include other and/or additional types of sensors for the detection of, for instance, barometric pressure, humidity, outdoor temperature, etc. In some embodiments, GNSS functionality may be achieved via the communications circuit 36 or other circuits coupled to the processing circuit 26.

The signal conditioning circuits 24 include amplifiers and filters, among other signal conditioning components, to condition the sensed signals including data corresponding to the sensed physiological parameters and/or location signals before further processing is implemented at the processing circuit 26. Though depicted in FIG. 2 as respectively associated with each sensor 22, in some embodiments, fewer signal conditioning circuits 24 may be used (e.g., shared for more than one sensor 22). In some embodiments, the signal conditioning circuits 24 (or functionality thereof) may be incorporated elsewhere, such as in the circuitry of the respective sensors 22 or in the processing circuit 26 (or in components residing therein). Further, although described above as involving unidirectional signal flow (e.g., from the sensor 22 to the signal conditioning circuit 24), in some embodiments, signal flow may be bi-directional. For instance, in the case of optical measurements, the microcontroller may cause an optical signal to be emitted from a light source (e.g., light emitting diode(s) or LED(s)) in or coupled to the circuitry of the sensor 22, with the sensor 22 (e.g., photocell) receiving the reflected/refracted signals.

The communications circuit 36 is managed and controlled by the processing circuit 26 (e.g., executing the communications software 34). The communications circuit 36 is used to wirelessly interface with the electronics device 14 (FIG. 3) and/or one or more devices of the computing system 20. In one embodiment, the communications circuit 36 may be configured as a Bluetooth transceiver, though in some embodiments, other and/or additional technologies may be used, such as Wi-Fi, GSM, LTE, CDMA and its derivatives, Zigbee, NFC, among others. In the embodiment depicted in FIG. 2, the communications circuit 36 comprises a transmitter circuit (TX CKT), a switch (SW), an antenna, a receiver circuit (RX CKT), a mixing circuit (MIX), and a frequency hopping controller (HOP CTL). The transmitter circuit and the receiver circuit comprise components suitable for providing respective transmission and reception of an RF signal, including a modulator/demodulator, filters, and amplifiers. In some embodiments, demodulation/modulation and/or filtering may be performed in part or in whole by the DSP. The switch switches between receiving and transmitting modes. The mixing circuit may be embodied as a frequency synthesizer and frequency mixers, as controlled by the processing circuit 26. The frequency hopping controller controls the hopping frequency of a transmitted signal based on feedback from a modulator of the transmitter circuit. In some embodiments, functionality for the frequency hopping controller may be implemented by the microcontroller or DSP. Control for the communications circuit 36 may be implemented by the microcontroller, the DSP, or a combination of both. In some embodiments, the communications circuit 36 may have its own dedicated controller that is supervised and/or managed by the microcontroller.

In one example operation, a signal (e.g., at 2.4 GHz) may be received at the antenna and directed by the switch to the receiver circuit. The receiver circuit, in cooperation with the mixing circuit, converts the received signal into an intermediate frequency (IF) signal under frequency hopping control attributed by the frequency hopping controller and then to baseband for further processing by the ADC. On the transmitting side, the baseband signal (e.g., from the DAC of the processing circuit 26) is converted to an IF signal and then RF by the transmitter circuit operating in cooperation with the mixing circuit, with the RF signal passed through the switch and emitted from the antenna under frequency hopping control provided by the frequency hopping controller. The modulator and demodulator of the transmitter and receiver circuits may perform frequency shift keying (FSK) type modulation/demodulation, though not limited to this type of modulation/demodulation, which enables the conversion between IF and baseband. In some embodiments, demodulation/modulation and/or filtering may be performed in part or in whole by the DSP. The memory 28 stores the communications software 34, which when executed by the microcontroller, controls the Bluetooth (and/or other protocols) transmission/reception.

Though the communications circuit 36 is depicted as an IF-type transceiver, in some embodiments, a direct conversion architecture may be implemented. As noted above, the communications circuit 36 may be embodied according to other and/or additional transceiver technologies.

The processing circuit 26 is depicted in FIG. 2 as including the ADC and DAC. For sensing functionality, the ADC converts the conditioned signal from the signal conditioning circuit 24 and digitizes the signal for further processing by the microcontroller and/or DSP. The ADC may also be used to convert analogs inputs that are received via the input interface 38 to a digital format for further processing by the microcontroller. The ADC may also be used in baseband processing of signals received via the communications circuit 36. The DAC converts digital information to analog information. Its role for sensing functionality may be to control the emission of signals, such as optical signals or acoustic signals, from the sensors 22. The DAC may further be used to cause the output of analog signals from the output interface 40. Also, the DAC may be used to convert the digital information and/or instructions from the microcontroller and/or DSP to analog signals that are fed to the transmitter circuit. In some embodiments, additional conversion circuits may be used.

The microcontroller and the DSP provide processing functionality for the wearable device 12. In some embodiments, functionality of both processors may be combined into a single processor, or further distributed among additional processors. The DSP provides for specialized digital signal processing, and enables an offloading of processing load from the microcontroller. The DSP may be embodied in specialized integrated circuit(s) or as field programmable gate arrays (FPGAs). In one embodiment, the DSP comprises a pipelined architecture, which comprises a central processing unit (CPU), plural circular buffers and separate program and data memories according to a Harvard architecture. The DSP further comprises dual busses, enabling concurrent instruction and data fetches. The DSP may also comprise an instruction cache and I/O controller, such as those found in Analog Devices SHARC® DSPs, though other manufacturers of DSPs may be used (e.g., Freescale multi-core MSC81xx family, Texas Instruments C6000 series, etc.). The DSP is generally utilized for math manipulations using registers and math components that may include a multiplier, arithmetic logic unit (ALU, which performs addition, subtraction, absolute value, logical operations, conversion between fixed and floating point units, etc.), and a barrel shifter. The ability of the DSP to implement fast multiply-accumulates (MACs) enables efficient execution of Fast Fourier Transforms (FFTs) and Finite Impulse Response (FIR) filtering. Some or all of the DSP functions may be performed by the microcontroller. The DSP generally serves an encoding and decoding function in the wearable device 12. For instance, encoding functionality may involve encoding commands or data corresponding to transfer of information to the electronics device 14 or a device of the computing system 20. Also, decoding functionality may involve decoding the information received from the sensors 22 (e.g., after processing by the ADC).

The microcontroller comprises a hardware device for executing software/firmware, particularly that stored in memory 28. The microcontroller can be any custom made or commercially available processor, a central processing unit (CPU), a semiconductor based microprocessor (in the form of a microchip or chip set), a macroprocessor, or generally any device for executing software instructions. Examples of suitable commercially available microprocessors include Intel's® Itanium® and Atom® microprocessors, to name a few non-limiting examples. The microcontroller provides for management and control of the wearable device 12, including determining physiological parameters or location coordinates based on the sensors 22, and for enabling communication with the electronics device 14 and/or a device of the computing system 20.

The memory 28 can include any one or a combination of volatile memory elements (e.g., random access memory (RAM, such as DRAM, SRAM, SDRAM, etc.)) and nonvolatile memory elements (e.g., ROM, Flash, solid state, EPROM, EEPROM, etc.). Moreover, the memory 28 may incorporate electronic, magnetic, and/or other types of storage media.

The software in memory 28 may include one or more separate programs, each of which comprises an ordered listing of executable instructions for implementing logical functions. In the example of FIG. 2, the software in the memory 28 includes a suitable operating system and the application software 30, which includes a plurality of software modules 32-34 for implementing certain embodiments of a messaging system and algorithms for determining physiological and/or behavioral measures and/or other information (e.g., including location, speed of travel, etc.) based on the output from the sensors 22. The raw data from the sensors 22 may be used by algorithms of the module 32 to determine various physiological and/or behavioral measures (e.g., heart rate, biomechanics, such as swinging of the arms), and may also be used to derive other parameters, such as energy expenditure, heart rate recovery, aerobic capacity (e.g., VO2 max, etc.), among other derived measures of physical performance. In some embodiments, these derived parameters may be computed externally (e.g., at the electronics devices 14 or one or more devices of the computing system 20) in lieu of, or in addition to, the computations performed local to the wearable device 12.

The operating system essentially controls the execution of computer programs, such as the application software 30 and associated modules 32-34, and provides scheduling, input-output control, file and data management, memory management, and communication control and related services. The memory 28 may also include user data, including weight, height, age, gender, goals, body mass index (BMI) that are used by the microcontroller executing the executable code of the algorithms to accurately interpret the measured physiological and/or behavioral data. The user data may also include historical data relating past recorded data to prior contexts. In some embodiments, user data may be stored elsewhere (e.g., at the electronics device 14 and/or a device of the remote computing system 20).

Although the application software 30 (and component parts 32-34) are described above as implemented in the wearable device 12, some embodiments may distribute the corresponding functionality among the wearable device 12 and other devices (e.g., electronics device 14 and/or one or more devices of the computing system 20), or in some embodiments, functionality of the application software 30 (and component parts 32-34) may be implemented in another device (e.g., the electronics device 14).

The software in memory 28 comprises a source program, executable program (object code), script, or any other entity comprising a set of instructions to be performed. When a source program, then the program may be translated via a compiler, assembler, interpreter, or the like, so as to operate properly in connection with the operating system. Furthermore, the software can be written as (a) an object oriented programming language, which has classes of data and methods, or (b) a procedure programming language, which has routines, subroutines, and/or functions, for example but not limited to, C, C++, Python, Java, among others. The software may be embodied in a computer program product, which may be a non-transitory computer readable medium or other medium.

The input interface(s) 38 comprises one or more interfaces (e.g., including a user interface) for entry of user input, such as a button or microphone or sensor (e.g., to detect user input) or touch-type display. In some embodiments, the input interface 38 may serve as a communications port for downloaded information to the wearable device 12 (such as via a wired connection). The output interface(s) 40 comprises one or more interfaces for the presentation or transfer of data, including a user interface (e.g., display screen presenting a graphical user interface) or communications interface for the transfer (e.g., wired) of information stored in the memory, or to enable one or more feedback devices, such as lighting devices (e.g., LEDs), audio devices (e.g., tone generator and speaker), and/or tactile feedback devices (e.g., vibratory motor). For instance, the output interface 40 may be used to present the messages to the user in some embodiments. In some embodiments, at least some of the functionality of the input and output interfaces 38 and 40, respectively, may be combined, including being embodied at least in part as a touch-type display screen for the entry of input and/or presentation of messages, among other data.

Referring now to FIG. 3, shown is an example electronics device 14 in which all or a portion of the functionality of a messaging system may be implemented. In the depicted example, the electronics device 14 is embodied as a smartphone (hereinafter, referred to as smartphone 14), though in some embodiments, other types of devices may be used, such as a workstation, laptop, notebook, tablet, etc. It should be appreciated by one having ordinary skill in the art that the logical block diagram depicted in FIG. 3 and described below is one example, and that other designs may be used in some embodiments. The application software 30A comprises a plurality of software modules (e.g., executable code/instructions) including position determining software (PDSW) 42, communications software (CMSW) 44, and messaging software (MSGSW) 46. In some embodiments, the application software 30A may include additional software modules or fewer software modules. The smartphone 14 comprises at least two different processors, including a baseband processor (BBP) 48 and an application processor (APP) 50. As is known, the baseband processor 48 primarily handles baseband communication-related tasks and the application processor 50 generally handles inputs and outputs and all applications other than those directly related to baseband processing. The baseband processor 48 comprises a dedicated processor for deploying functionality associated with a protocol stack (PROT STK) 52, such as a GSM (Global System for Mobile communications) protocol stack, among other functions. The application processor 50 comprises a multi-core processor for running applications, including all or a portion of the application software 30A and its corresponding component modules 42-46. The baseband processor 48 and application processor 50 have respective associated memory (e.g., MEM) 54, 56, including random access memory (RAM), Flash memory, etc., and peripherals, and a running clock. Note that, though depicted as residing in memory 56, all or a portion of the modules 42-46 of the application software 30A may be stored in memory 54, distributed among memory 54, 56, or reside in other memory.

More particularly, the baseband processor 48 may deploy functionality of the protocol stack 52 to enable the smartphone 14 to access one or a plurality of wireless network technologies, including WCDMA (Wideband Code Division Multiple Access), CDMA (Code Division Multiple Access), EDGE (Enhanced Data Rates for GSM Evolution), GPRS (General Packet Radio Service), Zigbee (e.g., based on IEEE 802.15.4), Bluetooth, Wi-Fi (Wireless Fidelity, such as based on IEEE 802.11), and/or LTE (Long Term Evolution), among variations thereof and/or other telecommunication protocols, standards, and/or specifications. The baseband processor 48 manages radio communications and control functions, including signal modulation, radio frequency shifting, and encoding. The baseband processor 48 comprises, or may be coupled to, a radio (e.g., RF front end) 58 and/or a GSM modem, and analog and digital baseband circuitry (ABB, DBB, respectively in FIG. 3). The radio 58 comprises one or more antennas, a transceiver, and a power amplifier to enable the receiving and transmitting of signals of a plurality of different frequencies, enabling access to the cellular network 16 (FIG. 1), and hence the communication of user data, activity data, and associated contexts to the computing system 20 (FIG. 1) and the receipt of or access to messaging from the computing system 20. The analog baseband circuitry is coupled to the radio 58 and provides an interface between the analog and digital domains of the GSM modem. The analog baseband circuitry comprises circuitry including an analog-to-digital converter (ADC) and digital-to-analog converter (DAC), as well as control and power management/distribution components and an audio codec to process analog and/or digital signals received indirectly via the application processor 50 or directly from the smartphone user interface (UI) 60 (e.g., microphone, earpiece, ring tone, vibrator circuits, etc.). The ADC digitizes any analog signals for processing by the digital baseband circuitry. The digital baseband circuitry deploys the functionality of one or more levels of the GSM protocol stack (e.g., Layer 1, Layer 2, etc.), and comprises a microcontroller (e.g., microcontroller unit or MCU, also referred to herein as a processor) and a digital signal processor (DSP, also referred to herein as a processor) that communicate over a shared memory interface (the memory comprising data and control information and parameters that instruct the actions to be taken on the data processed by the application processor 50). The MCU may be embodied as a RISC (reduced instruction set computer) machine that runs a real-time operating system (RTIOS), with cores having a plurality of peripherals (e.g., circuitry packaged as integrated circuits) such as RTC (real-time clock), SPI (serial peripheral interface), I2C (inter-integrated circuit), UARTs (Universal Asynchronous Receiver/Transmitter), devices based on IrDA (Infrared Data Association), SD/MMC (Secure Digital/Multimedia Cards) card controller, keypad scan controller, and USB devices, GPRS crypto module, TDMA (Time Division Multiple Access), smart card reader interface (e.g., for the one or more SIM (Subscriber Identity Module) cards), timers, and among others. For receive-side functionality, the MCU instructs the DSP to receive, for instance, in-phase/quadrature (I/Q) samples from the analog baseband circuitry and perform detection, demodulation, and decoding with reporting back to the MCU. For transmit-side functionality, the MCU presents transmittable data and auxiliary information to the DSP, which encodes the data and provides to the analog baseband circuitry (e.g., converted to analog signals by the DAC).

The application processor 50 operates under control of an operating system (OS) that enables the implementation of a plurality of user applications, including the application software 30A. The application processor 50 may be embodied as a System on a Chip (SOC), and supports a plurality of multimedia related features including web browsing functionality of the communications software 44 to access one or more computing devices of the computing system 20 (FIG. 4) that are coupled to the Internet, email, multimedia entertainment, games, etc. For instance, the application processor 50 may execute interface software of the communications software 44 (e.g., middleware, such as a browser with or operable in association with one or more application program interfaces (APIs)) to enable access to a cloud computing framework or other networks to provide remote data access/storage/processing, and through cooperation with an embedded operating system, access to calendars, location services, reminders, etc. For instance, in some embodiments, the messaging system may operate using cloud computing, where the processing of sensor data received (indirectly via the smartphone 14 or directly) from the wearable device 12 and context data (e.g., location data) and user inputted data received from the smartphone 14, including other sensed data from the smartphone (e.g., motion sense, accelerations, speed of travel, imaging, radio tag information (e.g., RFID), etc.), may be achieved by one or more devices of the computing system 20. The application processor 50 generally comprises a processor core (Advanced RISC Machine or ARM), and further comprises or may be coupled to multimedia modules (for decoding/encoding pictures, video, and/or audio), a graphics processing unit (GPU), communication interfaces (COMM) 62, and device interfaces. In one embodiment, the communication interfaces 62 may include wireless interfaces, including a Bluetooth (BT) (and/or Zigbee in some embodiments) module that enable wireless communication with an electronics device, including the wearable device 12, other electronics devices, and a Wi-Fi module for interfacing with a local 802.11 network, according to corresponding software in the communications software 44. The application processor 50 further comprises, or is coupled to, a global navigation satellite systems (GNSS) transceiver or receiver (GNSS) 64 for enabling access to a satellite network to, for instance, provide coordinate location services. In some embodiments, the GNSS receiver 64, in association with GNSS functionality in the application software 30A (e.g., as part of the position determining software (PDSW) 42, or in some embodiments, as a separate module), collects contextual data (time and location data, including location coordinates and altitude), and provides a time stamp to the information provided to a device or devices of the computing system 20. In some embodiments, the application software 30A may compute speed of movement of the smartphone 14 (and/or other sensor data, including acceleration data) for provision of the contextual information (e.g., meta information) to the remote computing system 20. For instance, the application software 30A may also collect information about the means of ambulation, where the GPS data (which may include time coordinates) may be used by the application software 30A to determine speed of travel, which may indicate whether the user is moving within a vehicle, on a bicycle, or walking or running. In some embodiments, other and/or additional data may be used to assess the type of activity, including physiological data (e.g., heart rate, respiration rate, galvanic skin response, etc.) and/or behavioral data.

The device interfaces coupled to the application processor 50 may include the user interface 60, including a display screen. The display screen, similar to a display screen of the wearable device user interface, may be embodied in one of several available technologies, including LCD or Liquid Crystal Display (or variants thereof, such as Thin Film Transistor (TFT) LCD, In Plane Switching (IPS) LCD)), light-emitting diode (LED)-based technology, such as organic LED (OLED), Active-Matrix OLED (AMOLED), or retina or haptic-based technology. For instance, the messaging software 46 may cooperate with the display screen to present web pages, dashboards, messages, and/or other documents or data received from the computing system 20 and/or the display screen may be used to present information (e.g., messages) in graphical user interfaces (GUIs) rendered locally in association with the messaging software 46. Other user interfaces 60 may include a keypad, microphone, speaker, ear piece connector, I/O interfaces (e.g., USB (Universal Serial Bus)), SD/MMC card, among other peripherals. Also coupled to the application processor 50 is an image capture device (IMAGE CAPTURE) 66. The image capture device 66 comprises an optical sensor (e.g., a charged coupled device (CCD) or a complementary metal-oxide semiconductor (CMOS) optical sensor). The image capture device 66 may be used to detect various physiological parameters of a user, including blood pressure based on remote photoplethysmography (PPG). Also included is a power management device 68 that controls and manages operations of a battery 70. The components described above and/or depicted in FIG. 3 share data over one or more busses, and in the depicted example, via data bus 72. It should be appreciated by one having ordinary skill in the art, in the context of the present disclosure, that variations to the above may be deployed in some embodiments to achieve similar functionality.

In the depicted embodiment, the application processor 50 runs the application software 30A, which in one embodiment, includes a plurality of software modules (e.g., executable code/instructions) including the position determining software 42, the communications software 44, and the messaging software 46. The position determining software 42 may include GNSS functionality that operates with the GNSS receiver 64 to interpret the data to provide a location and time of the user activity. As described above, the position determining software 42 provides location coordinates (and a corresponding time) of the user based on the GNSS receiver input. In some embodiments, the position determining software 42 cooperates with local or external location servicing services, wherein the position determining software 42 receives descriptive information and converts the information to latitude and longitude coordinates. In one embodiment, the communications software 44, in conjunction with the communications interface 62, enables the receipt, and/or communication of data to, the wearable device 12 (FIG. 2). The communications software 44 may enable operations according to any one or more of a variety of technologies, including BT, NFC, RFID, etc. The communications software 44 further includes network interfacing software, including browser software, to access the Internet (and in particular, one or more devices of the computing system 20). The messaging software 46 may render a GUI on the display screen (e.g., user interface 60) based on receipt of messaging information from the remote computing system 20. In one embodiment, the GUI may be locally generated, or in some embodiments, the GUI may comprise one or more web pages provided by the remote computing system 20, which include one or more messages intended at least in part to influence a change in behavior of the user, feedback, education material, and/or a dashboard pertaining to a (graphical) illustration of current and optionally historical activity of the user. In some embodiments, the dashboard may include population statistics used for comparative measures, which may be drawn from social websites according to one or more APIs or other databases (e.g., medical institution databases, coaching databases, user database(s), etc.). In some embodiments, one or more of the software modules and/or corresponding functionality of the application software 30A may be further distributed among additional software modules, or in some embodiments, performed at other devices in addition to, or in lieu of, implementation at the smartphone 14. The application software 30A may also comprises executable code to process the signals (and associated data) measured by the sensors (of the wearable device 12 as communicated to the smartphone 14, or based on sensors integrated within the smartphone 14) and record and/or derive physiological parameters, such as heart rate, blood pressure, respiration, perspiration, etc. Note that all or a portion of the aforementioned hardware and/or software of the smartphone 14 may also be referred to herein as a processing circuit in some embodiments.

Referring now to FIG. 4, shown is a computing device 74 that may comprise a device of the remote computing system 20 (FIG. 1) and which may comprise all or a portion of the functionality of a messaging system. Functionality of the computing device 74 may be implemented within a single computing device as shown here, or in some embodiments, may be implemented among plural devices (i.e., that collectively perform the functionality described below). In one embodiment, the computing device 74 may be embodied as an application server device, a computer, among other computing devices. One having ordinary skill in the art should appreciate in the context of the present disclosure that the example computing device 74 is merely illustrative of one embodiment, and that some embodiments of computing devices may comprise fewer or additional components, and/or some of the functionality associated with the various components depicted in FIG. 4 may be combined, or further distributed among additional modules or computing devices, in some embodiments. The computing device 74 is depicted in this example as a computer system, including one providing functionality of an application server. It should be appreciated that certain well-known components of computer systems are omitted here to avoid obfuscating relevant features of the computing device 74. In one embodiment, the computing device 74 comprises a processing circuit 76 comprising hardware and software components. In some embodiments, the processing circuit 76 may comprise additional components or fewer components. For instance, memory may be separate. The processing circuit 76 comprises one or more processors, such as processor 78 (PROCES), input/output (I/O) interface(s) 80 (I/O), and memory 82 (MEM), all coupled to one or more data busses, such as data bus 84 (DBUS). The memory 82 may include any one or a combination of volatile memory elements (e.g., random-access memory RAM, such as DRAM, and SRAM, etc.) and nonvolatile memory elements (e.g., ROM, Flash, solid state, EPROM, EEPROM, hard drive, tape, CDROM, etc.). The memory 82 may store a native operating system (OS), one or more native applications, emulation systems, or emulated applications for any of a variety of operating systems and/or emulated hardware platforms, emulated operating systems, etc. In some embodiments, the processing circuit 76 may include, or be coupled to, one or more separate storage devices. For instance, in the depicted embodiment, the processing circuit 76 is coupled via the I/O interfaces 80 to user profile data structures (UPDS) 86 and messaging data structures (MSDS) 88, explained further below. In some embodiments, the user profile data structures 86 and messaging data structures 88 may be coupled to the processing circuit 76 directly via the data bus 84 (e.g., stored in a storage device (STOR DEV)) or coupled to the processing circuit 76 via the I/O interfaces 80 and the network 18 via one or more network-connected storage devices. In some embodiments, the user profile data structures 86 and messaging data structures 88 may be stored in a single device or distributed among plural devices. Though described as separate data structures, in some embodiments, the content stored by the user profile data structures 86 and messaging data structures 88 may be combined into a single data structure. The user profile data structures 86 and messaging data structures 88 may be stored in persistent memory (e.g., optical, magnetic, and/or semiconductor memory and associated drives). In some embodiments, the user profile data structures 86 and messaging data structures 88 may be stored in memory 82. The user profile data structures 86 are configured to store user profile data. In one embodiment, the user profile data comprises demographics and user responses to intake questionnaires. In some embodiments, the user profile data may include responses to on-going questions presented at the smartphone 14 (e.g., presented to the user daily, weekly, bi-weekly, etc.). For instance, user awareness, as described further below, may be based on questions presented to the user via the smartphone 14 every couple of weeks. The intake questionnaires may be presented, for instance, as an on-line questionnaire during registration, where questions are asked of the user including significant locations of the user, travel options between those significant locations, questions about the user's motivation, skills, barriers, goals, outcome expectations. The user profile data structures 86 may be accessed by the processor 78 executing software in memory 82 to determine coaching domains and/or facilitate message selection. In some embodiments, one or more of the content stored in the user profile data structures 86 may also be stored as meta information in the messaging data structures 88. The user profile data structure 86 may also include current or contemporaneous activity data for the user that is communicated to the computing device 74 during synch operations with the smartphone 14 and/or wearable device 12 or as communicated from a third party server device (e.g., medical facility, fitness tracking service, etc.). Additional data structures may be used to record similar information for other users. The messaging data structures 88 are described further below in conjunction with FIG. 8, but in general, provide an association of personal determinants for a user with a plurality of messages and include other meta information that enable a determination as to a proper context to present a given message. The messaging data structures 88 may be maintained by an administrator operating the computing system 20 and/or computing device 74. In some embodiments, the user profile data structures 86 and messaging data structures 88 may serve as backend storage of the computing system 20 as well as network storage and/or cloud storage. The user profile data structures 86 and messaging data structures 88 are updated periodically, aperiodically, and/or in response to a request from the wearable device 12, the electronics device 14, and/or the operations of the computing device 74.

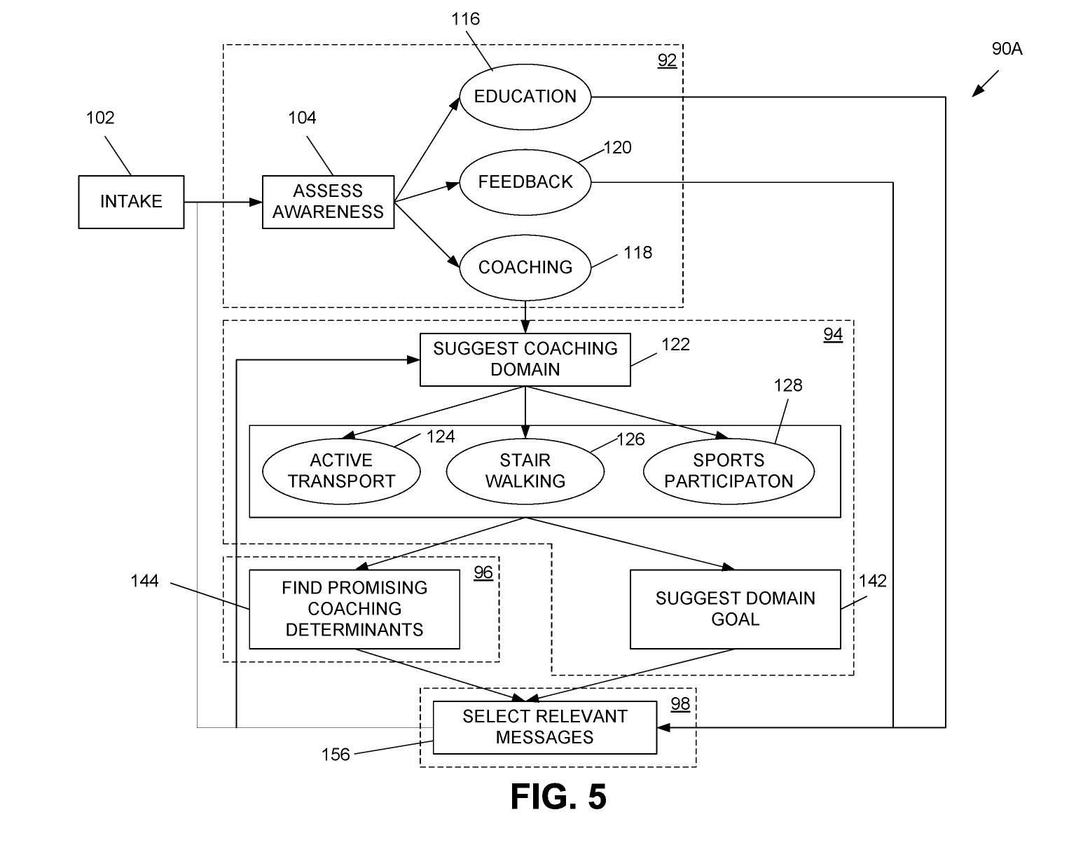

In the embodiment depicted in FIG. 4, the memory 82 comprises an operating system (OS) and application software (ASW) 30B. The application software 30B comprises a reasoning engine 90, which in one embodiment, comprises instructions or software modules (e.g., executable code) including awareness/activity level assessor software (AALASW) 92, domain determiner software (DDSW) 94, personal determinant finder software (PDFSW) 96, and messaging selection and tailoring software (MSTSW) 98. In general, the reasoning engine 90 receives input that includes answers to intake questions, measurements from the wearable device 12 and/or smartphone 14, and user input (e.g., from questions prompted by the reasoning engine 90 daily, weekly, among other periodic or aperiodic intervals). The information may be stored in and accessed by the reasoning engine 90 from the user profile data structures 86 and/or messaging data structures 88, or in some embodiments, accessed from memory 82 or other storage devices internal or external to the computing device 74. Although some of the inputs to the reasoning engine 90 are described in the context of some user-intervention, it should be appreciated that one or more of the inputs may be collected automatically and/or derived by learning algorithms. For instance, the home location may be derived by GNSS data and recorded time durations at given locations that meet or exceed a threshold value, since it is a reasonable assumption that a given location where a user spends the majority of his or her time during evenings and weekends is likely the home location. Also, transport options may be retrieved by the reasoning engine 90 running or invoking Internet bots that search route planning websites. Further, by monitoring a number of floors a user climbs while at a location, possibly with changes in monitored altitude, the reasoning engine 90 may run (or invoke) an algorithm that estimates the number of floors that can be climbed by the user. Note that in some embodiments, functionality of the reasoning engine 90 may be implemented in part or in whole at alternative devices, including at the wearable device 12 and/or the electronics devices 14. The memory 82 further comprises a communications module (COMM MODULE) 100 that enables communications among network-connected devices and provides web and/or cloud services, among other software such as via one or more APIs. In some embodiments, the communications module 100 may be a part of the application software 30B. In one example operation, the communications module 100 may receive (via I/O interfaces 80) input data (e.g., a content feed) from the wearable device 12 and/or the electronics device 14 that includes sensed data, context data, user-inputted data, data from third-party databases (e.g., medical data base, health program provider data, etc.), data from social media, data from questionnaires, data from external devices (e.g., weight scales, environmental sensors, etc.), among other data. The content feed may be continual, intermittent, and/or scheduled. The communications module 100 may, in one embodiment, provide messages, which may include other information such as activity monitoring data and/or other dashboard data, via I/O interfaces 80 to the wearable device 12 and/or the electronics device 14. In some embodiments, the communications module 100 may comprise a web service component or cloud component which is accessed by a client application (e.g., browser) residing at the wearable device 12 and/or electronics device 14. Functionality of the reasoning engine 90 is described below in conjunction with FIG. 5.

Execution of the application software 30B (including the reasoning engine 90 and associated software modules 92-98) and communications software 100 may be implemented by the processor 78 under the management and/or control of the operating system. The processor 78 may be embodied as a custom-made or commercially available processor, a central processing unit (CPU) or an auxiliary processor among several processors, a semiconductor based microprocessor (in the form of a microchip), a macroprocessor, one or more application specific integrated circuits (ASICs), a plurality of suitably configured digital logic gates, and/or other well-known electrical configurations comprising discrete elements both individually and in various combinations to coordinate the overall operation of the computing device 74.

The I/O interfaces 80 comprise hardware and/or software to provide one or more interfaces to the Internet 18, as well as to other devices such as a user interface (UI) (e.g., keyboard, mouse, microphone, display screen, etc.) and/or the data structures 86-88. The user interfaces may include a keyboard, mouse, microphone, immersive head set, display screen, etc., which enable input and/or output by an administrator or other user. The I/O interfaces 80 may comprise any number of interfaces for the input and output of signals (e.g., analog or digital data) for conveyance of information (e.g., data) over various networks and according to various protocols and/or standards. The user interface (UI) is configured to provide an interface between an administrator or content author and the computing device 74. The administrator may input a request via the user interface, for instance, to manage the user profile data structures 86 and/or the messaging data structures 88. Updates to the data structures 86 and/or 88 may also be achieved without administrator intervention.

When certain embodiments of the computing device 74 are implemented at least in part with software (including firmware), as depicted in FIG. 4, it should be noted that the software (e.g., including the application software 30B (and associated modules 90-98) and communications software 100) can be stored on a variety of non-transitory computer-readable medium for use by, or in connection with, a variety of computer-related systems or methods. In the context of this document, a computer-readable medium may comprise an electronic, magnetic, optical, or other physical device or apparatus that may contain or store a computer program (e.g., executable code or instructions) for use by or in connection with a computer-related system or method. The software may be embedded in a variety of computer-readable mediums for use by, or in connection with, an instruction execution system, apparatus, or device, such as a computer-based system, processor-containing system, or other system that can fetch the instructions from the instruction execution system, apparatus, or device and execute the instructions.

When certain embodiments of the computing device 74 are implemented at least in part with hardware, such functionality may be implemented with any or a combination of the following technologies, which are all well-known in the art: a discrete logic circuit(s) having logic gates for implementing logic functions upon data signals, an application specific integrated circuit (ASIC) having appropriate combinational logic gates, a programmable gate array(s) (PGA), a field programmable gate array (FPGA), relays, contactors, etc.

With continued reference to FIG. 4, attention is directed to FIG. 5, which illustrates an example process 90A implemented by a messaging system, and in particular, implemented by an embodiment of the reasoning engine 90. With reference to the functionality of the awareness/activity level assessor software 92, intake 102 (e.g., activity data, user profile data, including user input from ongoing (e.g., periodic) questions) is received and the user's awareness is assessed 104. The user's awareness is assessed based on two evaluations of the user's physical activity level, namely an objective evaluation (e.g., whether the user meets a norm or target) and a subjective evaluation (e.g., whether the user thinks he or she is sufficiently physically active). In one embodiment, the objective evaluation is based on the physical activity data for a defined period (e.g., the past week). For instance, functionality of the awareness/activity level assessor software 92 evaluates the number of minutes spent on sports activities (e.g., based on user logs) and the weekly number of steps (based on measurements obtained via the wearable device 12). If one or both of these parameters are above a defined threshold (e.g., 150 minutes of sports and/or 70,000 steps), the awareness/activity level assessor software 92 determines that the physical activity level is objectively sufficient. In one embodiment, the subjective evaluation is based on user input, which may be assessed via one or more questions delivered through the smartphone 14. For instance, a question may be presented that asks what the user thinks of his or her own physical activity level, and he or she can answer with more than sufficient, sufficient, insufficient, or very insufficient. Note that other types or degrees of reply options may be used in some embodiments. Continuing the example, if the user chooses one of the first two options, his or her physical activity is considered subjectively sufficient. If the user chooses one of the last two options, his or her physical activity is subjectively insufficient. The awareness assessment is repeated every defined (e.g., predetermined) number weeks, and the periodicity (or in general, repeat frequency or timing) can change over time. In one embodiment, awareness questions may be repeated every 3 weeks, though a greater or lesser repeat frequency may be used in some embodiments, and in some embodiments, may be aperiodic (e.g., event or condition based). The objective measurements may be continual (e.g., through each day). In one embodiment, the awareness/activity level assessor software 92 receives input (intake 102) in the form of measured activity (via the wearable device 12, smartphone 14, including steps), user input of sports activities through, for instance, the smartphone 14, and user input of physical activity evaluation through the smartphone 14.

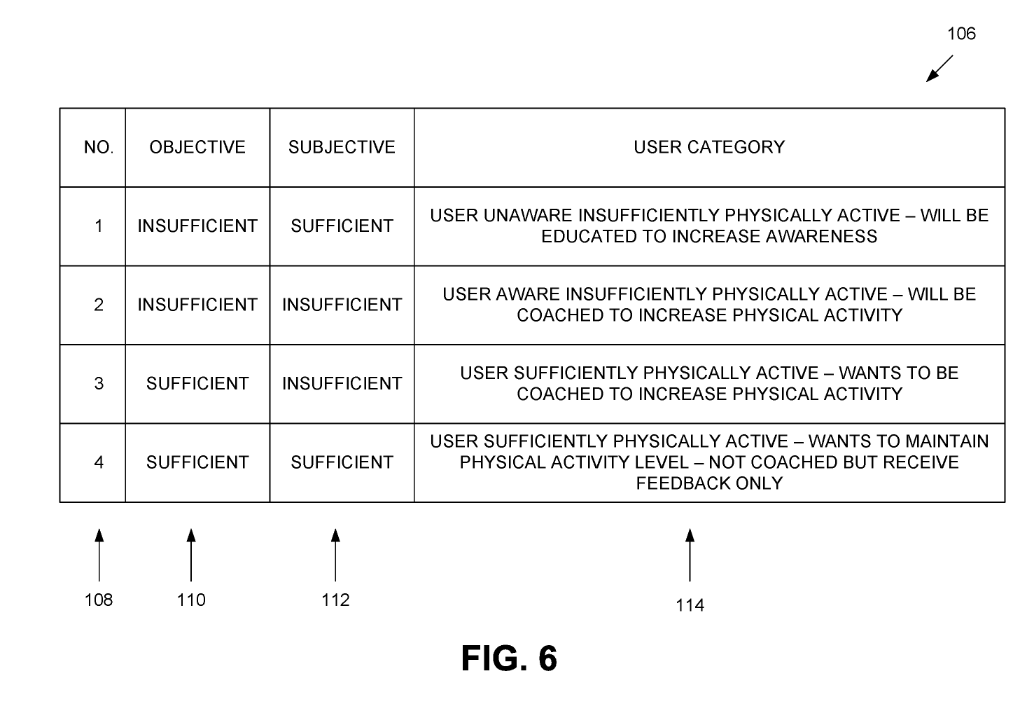

The rules-based approach implemented by the awareness/activity level assessor software 92 is concise and computationally efficient, and serves to facilitate a tailored or personalized approach to the type of influence a user needs to change activity patterns or behavior. An output of the assessment is an assignment of a user to one of four categories, which determines what type of coaching messages the user will (and will not) receive. Stated otherwise, based on the awareness assessment, the process 90A determines whether the messaging is provided according to either a coaching category 118 of messaging, a feedback category 120 of messaging, or an education category 116 of messaging. The four user categories (of awareness) are summarized in the table 106 illustrated in FIG. 6. Referring to the table 106, shown is a rule number column 108 (“No.”) numbered by rows from 1-4, an objective column 110 that takes on a logical value for each row (e.g., insufficient or sufficient), a subjective column 112 that likewise takes on a logical value for each row (e.g., insufficient or sufficient), and a user category or rule 114. The objective column 110 is based on a given standard or norms (e.g., global, governmental, industry, and/or other norms), the subjective column 112 indicates a perspective of the user (e.g., does the user think that he or she is sufficiently active or not). Referring to rule 1, the user category is that the user is unaware that he or she (hereinafter, reference is to the male gender for brevity, with the understanding that the female gender may likewise apply) is insufficiently physically active (objective equals insufficient), and will be educated (education category 116, FIG. 5) to increase his awareness. In other words, the user is insufficiently active (from the objective column 110), such as according to certain norms, but the user believes that he or she is sufficiently active (from the subjective column 112). Thus, the user in rule 1 needs to be educated about physical activity norms and the benefits of an active lifestyle, etc. Referring to rule 2, the user is aware that he is insufficiently physically active and will be coached (coaching category 118, FIG. 5) to increase his physical activity level. Referring to the third rule, the user is sufficiently physically active yet still wants to be coached (coaching category 118, FIG. 5) to increase his physical activity level. The fourth rule requires the user to be sufficiently physically active, and he wants to maintain his physical activity level. This user will not be coached to increase his physical activity level, but only receive feedback (feedback category 120, FIG. 5).

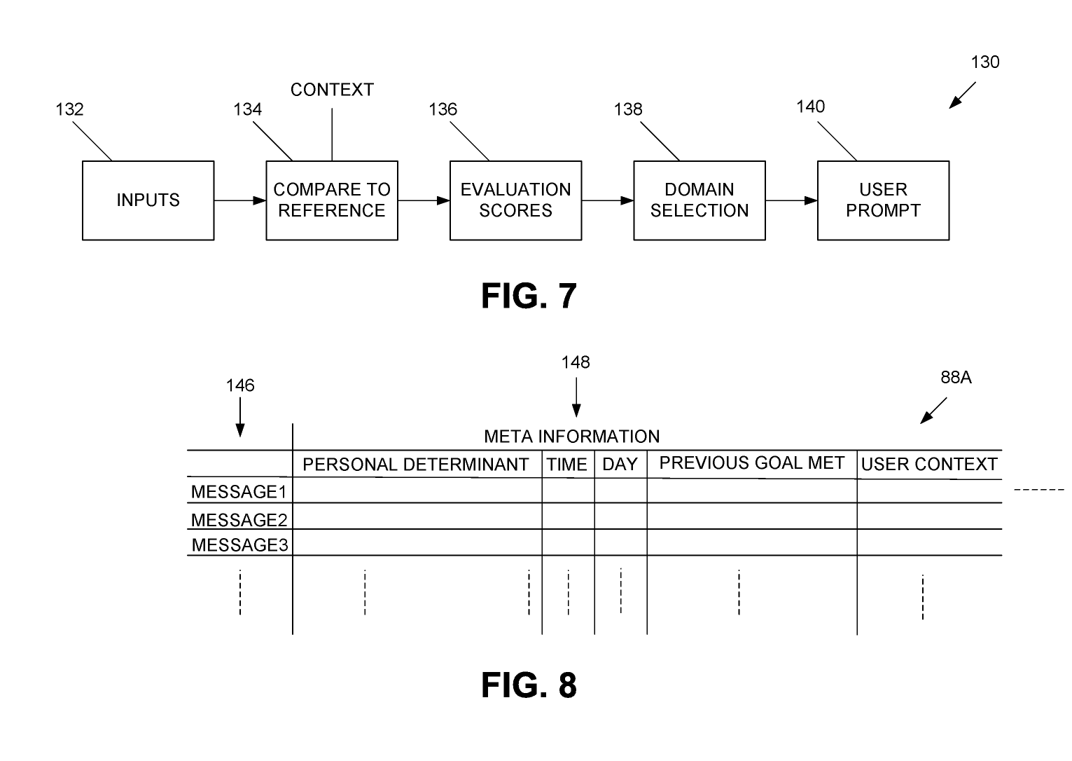

Referring again in particular to FIG. 5, attention is now directed to functionality of the domain determiner software 94. Users that are assigned to the coaching category (118) of messaging are guided (122) by the domain determiner software 94 to choose one of a plurality of physical activities or domains (e.g., active transport (124), stair walking (126), or sports participation (128)) to focus their behavior change efforts on. To do so, the domain determiner software 94 suggests (recommends) a domain or similarly, physical activity, based on a personal evaluation of the user's performance in these domains 124-128. Referring to the flow diagram 130 of FIG. 7 with continued reference to FIGS. 4-5, the user's physical activity in each of the possible domains 124-128 is estimated based on plural inputs (132) being received by the domain determiner software 94. In one embodiment, the inputs (132) comprise any one or a combination of activity data collected through the smartphone 14 (FIG. 3) and/or an activity monitor (e.g., the wearable device 12, FIG. 2, for instance receiving location (e.g., GNSS) data, stair climbing performance data, etc.) or through daily user input in an app (e.g., the application software 30A running on the smartphone 14, for instance receiving user input of sports activities, sports goals, and/or transport usage, reported locations and transport options through an intake questionnaire, reported locations and available stairs through an intake questionnaire, etc.). For instance, periodically (e.g., every day, twice per week, etc.), a number of questions may be posed to the user via the application software 30A of the smartphone 14. Information about the visited locations is used to prompt the user about travel options that he has used to go there. In some embodiments, daily questions are presented to the user via the application software 30A of the smartphone 14, which is used to solicit user input about visited locations and travel options between locations.

From the input (132), as suggested above, the domain determiner software 94 is aware of the activity levels of the different travel options for each user, and the domain determiner software 94 can derive the types of transport used and the number of active travel minutes. Note that the user may similarly be prompted by the smartphone application software 30A about the sports activities during the day before. When a user regularly answers that he did not participate in sporting activities, the frequency of asking about sports may be decreased to once per week or some other interval. The domain determiner software 94 computes a score for the user's behavior in each domain. In one embodiment, the score is based on the user's physical activity in that domain and a reference (reference point). Referring again to the flow diagram 130, the physical activity values are evaluated by comparing them to a reference (134). For instance, the reference may comprise estimated “maximum” or “ideal” values that are based on information about the user's context and visits to their important locations. As suggested above, this context information may be collected through a questionnaire (e.g., presented on-line and accessed by the smartphone application software 30A), and includes information about the addresses of significant locations, (active and non-active) travel options between those locations, relevant floor numbers on these locations, and the availability of stairs. Using these personal evaluations, the domain determiner software 94 determines evaluation scores (136). For instance, the domain with the largest potential for improvement can be detected, as the evaluation score for that domain will be lowest. Stated otherwise, for each of the domains, the domain determiner software 94 computes a numerical score, where each domain calculation may slightly differ. The scores are generally kept in the same range (e.g., between 0 and 1, though some embodiments may scale the scores differently).

Explaining further the processing by the domain determiner software 94 for each domain, in the sports domain (sports participation 128, FIG. 5), the user's physical activity is based on user logs of sports activities through repeated (daily, weekly, etc.) questions on the smartphone 14. In one embodiment, the reference point is the user's most recent goal for the sports domain. If he has never set a goal, a default goal may be chosen (e.g., 90 minutes per week). The user is asked to set a goal for the sports domain if he has opted for that domain, which happens through a question on the smartphone 14. For active transport (e.g., active transport 124, FIG. 5), the user's physical activity is based on user logs of transport to his visited locations. For instance, questions may be prompted by the user visiting his significant locations, thus in one embodiment using a combination of locations reported in the intake questionnaire and location (GNSS) data showing that he was at one of these locations. In the intake questionnaire, users may report, for instance, two transport options to each location: one more active and one less active in terms of minutes spent walking/biking. When logging transport activities, the user may choose between his more active and less active option. From those logs, the domain determiner software 94 computes a sum of minutes spent walking or biking to these locations. In one embodiment, the reference point may be calculated by assuming that the user opted for his more active transport option to each of his visited locations. Thus, the domain determiner software 94 may calculate the sum of minutes the user could have spent walking or biking. For stair walking (stair walking 126, FIG. 5), the user's physical activity may be based on the weekly number of stairs climbed as monitored through the wearable device 12 (FIG. 1) and communicated to the reasoning engine 90. In one embodiment, the reference point is again based on the significant locations that the user visited (based on locations reported in the intake questionnaire and GNSS data). For each location reported in the intake questionnaire, the user may be asked about relevant floors and the availability of stairs. More specifically, the smartphone app may ask at what floor the user usually has to be (e.g., on what floor do you work, live, work out, etc.), and how many extra floors he usually climbs when at that location. The domain determiner software 94 may take these two numbers (summed for all locations visited), compare it to the actual number of stairs climbed, and return a numerical score.

As some illustrative examples, for the sports participation domain (128), the calculation of scores may comprise, in one embodiment, a division operation between the actual behavior of the user and a personal user goal or other standard (e.g., corresponding to weekly minutes spent on sports). In this example, the score is calculated as 135 min/180 min=0.75. As another example, for the stair walking domain (126) and active transport domain (124), more information about the context of the user may be considered in the scoring. Using an example illustration for stair walking (126), the scoring may depend on significant locations that the user has visited in the past week (e.g., home, work, school, etc.) and the availability of stairs at those locations. With such information, an estimate of the number of floors that the user could climb is estimated and compared with the actual number of floors climbed by the user for that week using, for instance, a sigmoid function. Using an example illustration for active transport (124), the number of minutes that the user could have spent on active transport (e.g., based on locations the user visited and transport options for this location) is estimated and compared to the actual number of minutes the user has spent on active transport via a division operation. Also, the scores may be adjusted based on the number of repeated locations for which the user does not have multiple transport options. One skilled in the art should appreciate that the examples for scoring are illustrative of some examples, and that variations to the above may be discerned based on the present disclosure and are contemplated to be within the scope of the disclosure.

Continuing, the scores are compared, and the domain determiner software 94 selects a domain based on the evaluation scores (138) (e.g., the lowest score domain), and prompts the user (140). The prompt comprises a suggestion to the user of the selected domain as a focus for the coaching in the upcoming week. However, the user is allowed to overrule this suggestion and opt for another domain.

Referring again in particular to FIG. 5, after or in some embodiments as part of the prompt (140), the domain determiner software 94 prompts the user to set a specific goal for the coaching domain (142). The goal may be configured as weekly time spent on active transport, weekly time spent on sports or daily number of stairs climbed, among other formats or periods/quantities of activity. If a user meets the previous goal (e.g., in this coaching domain), the domain determiner software 94 suggests to the user to increase his goal by, for instance, 10%. If the user did not meet the goal last time, the domain determiner software 94 advises the user to keep the goal at the same level. Again, in order to ensure the user's autonomy, the final decision on the goal is up to the user.

Referring now to functionality of the personal determinant finder software 96 and FIG. 5, once the coaching domain is selected, the personal determinant finder software 96 investigates on which personal determinants the coaching messages should focus to yield the most promising effect on the desired behavior (144). Referring to FIG. 8, shown is a portion of one of the messaging data structures 88A for a given user (the messaging data structures 88 previously denoted in FIG. 4). The messaging data structure 88A comprises a large collection of coaching messages (146) that are each associated with constraints in meta information (148). For instance, one of the categories of those constraints is the personal determinant that is targeted, enabling the targeting of a subset of messages that comprise self-efficacy, for instance. A similar mechanism may be employed for the other constraints, which includes the time of day that is relevant for the message, the day of the week, whether a previous goal has been met, and other user contexts. For instance, a message or messages may be selected that may be sent on Sunday, or all messages that may be sent to user that have achieved their goal in the prior week, or all messages that are relevant to users that indicate they often feel too tired to do sports, among other examples. Stated generally, the messaging data structure 88A associates each of the messages 146 to meta information 148, which facilitates the determination by the personal determinant finder software 96 as to which circumstances are relevant for a given message. The messages are based on established behavior change techniques, including prompting barrier identification, providing information on consequences, and prompting goal setting, while also taking into account user preferences. The messages are crafted (e.g., by a content author, administrator, etc.) to be motivational, personally relevant, and trustworthy, and using the meta information, are annotated with restrictions for the circumstances under which the messages are relevant (e.g., day and time, the user's awareness phase and coaching domain, the user's perceptions reported in the intake questionnaire). Note that the structure illustrated for the messaging data structure 88A is illustrative of one example data structure, and that in some embodiments, other structures for associating the messages with the context information may be used. Also, the meta information is illustrative as well, and additional meta information may be included, including the user's occupational status, answers to questions in the intake questionnaire, the current weather (retrieved from online weather data), among other contextual information.

To determine what messages are most likely to positively affect the user's behavior, the personal determinant finder software 96 estimates the effects of improving each one of the personal determinants based on running simulations of a dynamic computational model. The computational model is a formalization of the dynamics between the personal determinants and the behavior, where each of the concepts is represented by a numerical value in range [0, 1]. For the model simulations, the personal determinant finder software 96 uses the initial values of the psychological concepts in the model, as well as the initial value of the behavior concept. For the psychological concepts, input may be based on the use of one question (with Likert scale answer options) per concept to assess its value. The presentation of questions may be performed repeatedly (e.g., weekly) through the smartphone 14. For the behavior concept, the personal determinant finder software 96 takes the behavior score for this domain, as calculated in the coaching determination step described above. In some embodiments, and for practical reasons, if this value is >1.0, it is capped off at the maximum of 1.0. Generally, the input used in the simulation process includes user input on psychological concepts acquired through the smartphone 14 and behavior scores as assessed for coaching domain determination.

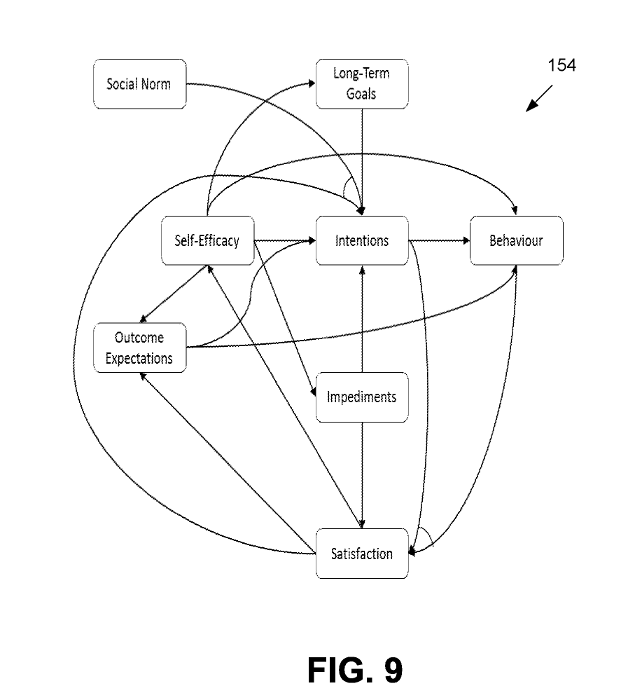

FIG. 9 provides a graphical representation of a computational model 154 used in one embodiment for the simulations. It is an adaptation of the computational model presented in the publication, “A Computational Agent Model of Influences on Physical Activity,” by Julia S. Mollee & C. Natalie Van der Wal, PRIMA 2013, LNAI 8291, pp. 478-485, 2013), and incorporated herein by reference. The computational model 154 describes dynamic relationships between all the concepts depicted in FIG. 9 (e.g., social norms, long-term goals, self-efficacy, intentions, behavior, outcome expectations, impediments, and satisfaction), which are formalized with differential equations as disclosed in the above-referenced publication and modelled numerically as real values between [0, 1]. The simulations are performed to assess the effects of specific behavior change strategies for a given user, and uses up-to-date input values (e.g., responses to daily or weekly questions presented to the user) for the personal determinants as a basis for the computations. The differences between the published model and the model 154 includes (a) the concept of long term goals is added, (b) the three types of outcome expectations (physical, personal, social) in the original model are aggregated into one overall concept of outcome expectations, (c) the concept of personal norm is removed, (d) the concept of social norm is changed to influencing the intentions directly, rather than through the (social) outcome expectations, and (e) the self-regulation skills are added as an explicit concept, rather than as an implicit parameter in the original model. The model 154 is mainly based on social cognitive theory, which describes the reasons why people fail or succeed to exhibit some desired (health) behavior from both social and cognitive determinants. The model 154 may be additionally based on other theories (e.g., self-regulation theory, health action process approach, etc.). Though the computational model 154 comprises eight (8) determinants, in some embodiments, fewer or a greater number of determinants may be used.

In one embodiment, the simulation process executed by the personal determinant finder software 96 begins by the estimating the current states of the personal determinants by means of short questions via the app (e.g., application software 30A) run on the smartphone 14 (FIG. 3) and communicated to the reasoning engine 90. The state refers to how strongly the personal determinant is present in the user (e.g., how strong are the user's intentions, how high is the user's sense of efficacy, etc.). The personal determinants are represented as concepts in the computational model 154 (FIG. 9), and have numbered values between 0 and 1 as set forth above. The resulting values are used as input for the computational model. To simulate the effect of targeting one of the personal determinants, one of the values obtained from the responses to the app questionnaire is increased according to the hypothesized effect of sending coaching messages about this determinant. The hypothesized increase is a relative boost of a defined percentage (e.g., 5%) of the difference to the maximum value (e.g., one (1)) for a first period of time for a given interval (e.g., first three (3) days of seven days) of the simulation. For instance, for the concept of self-efficacy, and using the formulas of the aforementioned publication, if (target==SE): SE(t)=SE(t)+0.05*(1−SE(t)). Then, the computational model simulates the dynamics between the determinants and estimates the effect on the behavior. Note that the behavior is a concept in the computational model. The personal determinants are the factors (e.g., psychological factors) that influence the behavior. One aim of certain embodiments of a messaging system is to improve behavior, and thus the interest in the value (e.g., predicted value) of the behavior in the simulations. By running simulations for each possible targeted personal determinant, a list of personal determinants is constructed, ordered by the most promising effect on the behavior variable. One outcome of the simulations is the predicted behavior value for each of the hypothesized effects of targeting one of the personal determinants. This order is taken into account when selecting coaching messages to the user. As with the selection of a coaching domain and goal, the simulation cycle is repeated (e.g., periodically, including weekly, or according to other periodic or aperiodic intervals) in order to tailor to the user's strongest psychological needs at all times. In one embodiment, in contrast to the evaluation of the user's behavior for suggesting a coaching domain, the simulation part of the reasoning engine 90 does not tailor the intervention (messaging) based on information about the user's environment. Rather, the intervention is tailored based on the user's motivational state of mind, enabling support on aspects that are relevant to the motivation and behavior of the user.

Referring now to the messaging selection and tailoring software 98 and FIG. 5, at given moments in time, the messaging selection and tailoring software 98 checks for messages that are relevant to send to the user (156). In one embodiment, selecting a message is based on elimination: starting from the set of all messages (e.g., from messaging data structure 88A, FIG. 8), the selection is narrowed down by filtering inapt or irrelevant messages. As explained above, the user is already assigned an awareness phase and has chosen a coaching domain. In addition, a coaching determinant is picked from the ordered list, with a probability relative to its position in the list. For instance, as illustrated in FIG. 9, there are eight possible determinants that may be targeted by the messages, so eight positions occupy the ordered list. A probability is assigned to each of the positions on the list, in one embodiment going from high to low with a total value of one (1). Each time a message is to be determined, a determinant is selected form the list according to the probabilities. In this way, determinants with the best simulation outcomes are most likely to be targeted by the messages, yet other messages are not completely ruled out. That is, this probability is introduced to increase diversity in the messages during the week. Then, all messages that are aimed at other awareness phases, coaching domains or targeted coaching determinants are filtered out. The messaging selection and tailoring software 98 checks for other aspects as well, including context from the meta information (e.g., meta information 148, FIG. 8), including the day and time, the user's occupational status, answers to questions in the intake questionnaire, whether the user is on track to reach their goal and the current weather (retrieved from online weather data). In general, to select a message, all messages that are irrelevant to the user based on different types of information are filtered out. This includes the user's current awareness phase and his selected coaching domain, answers to questions in the intake questionnaire, and the current day/time. Some messages may only be sent if the user is on track to reach his weekly goal, so for those instances, input includes the physical activity measurements (from the wearable device 12 and/or the smartphone 14, including user logs accessed through the smartphone). In addition, some messages may only be meant to be sent if the weather forecast is good (or vice versa), which information is also included as input (e.g., retrieved from an online weather service). In other words, in one embodiment, input to the messaging selection and tailoring software 98 may include answers to intake questionnaire, measured steps/stairs, user input of transport/sports activities through the smartphone 14, and/or online weather information.

Once a message is selected, if there are open fields, the open fields are filled in to tailor the message to the user and context. In one embodiment, to increase relatedness, most messages address the user with their first name. Additionally, some messages are completed by filling out the user's current daily number of steps or stairs, their accumulated weekly time spent on sports or active transport, their weekly goal, the percentage of their weekly goal they have reached so far, the maximum number of stairs they would consider to walk, and the current weather score.

Note that variations to operations of the aforementioned embodiments for the reasoning engine 90 may be used, including automatically detecting active transport versus logging transport based on two answer options, and/or detecting/learning significant locations over time versus reporting in an intake questionnaire, among other variations.

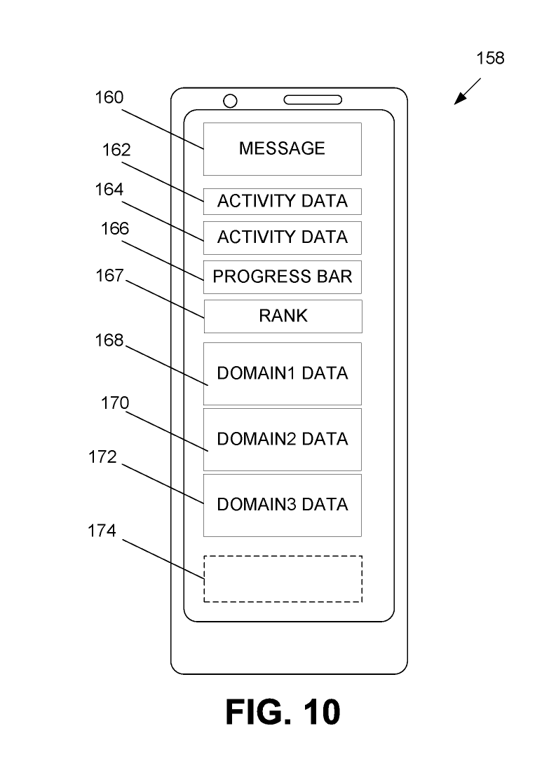

FIG. 10 provides a screen diagram that illustrates an example user interface 158 used by an embodiment of the messaging system in providing messaging to a user. The user interface 158 may be presented on the wearable device 12 and/or the electronics device 14. The user interface 158 may be presented as a web page or rendered according to local user interface functionality at the respective devices 12, 14. The user interface 158 comprises one or more graphical and/or textual components, including a message 160, activity data 162, 164, a progress bar 166, rank data 167, domain data 168, 170, and 172, and message 174. In one embodiment, the message 160 comprises a greeting from a virtual coach (e.g., avatar), including a reminder of the domain and goal selected for that week. The message 174, on the other hand, is a message that is periodically (or aperiodically in some embodiments) sent to the user as accessed from the messaging data structure (e.g., messaging data structure 88A, FIG. 8) and presented in the user interface 158 to target a personal determinant to better influence a change in behavior or physical activity for the user. The dash outline of the message 174 represents the temporary presentation (e.g., for a brief period of time during a given day, such as on a weekly or bi-weekly basis) of the messaging, as its presentation is contextual. Though shown at the lower portion of the user interface 158, in some embodiments, the message 174 may be rendered elsewhere on the user interface 158 or according to a different mechanism (e.g., may be overlaid on a dashboard and closed to continue the dashboard functionality). The message 174 (and message 160) may include text alone, or in some embodiments, any one or combination of text, graphics and/or video. Note that variations to the content of the user interface 158 may be used, including fewer components, more components (e.g., a list of the most recent messages), or different information. The message 174 comprises a textual message selected and tailored by the messaging selection and tailoring software 98 and communicated by the communications module 100. The messages 174 may be presented one or more times during a 24 hour period.

The design layout and/or content of the user interface 158 may differ depending on the selected domain. For instance, feedback and/or education-focused user interfaces may take on a different form and/or substance than the user-interface 158 dedicated to the coaching domain. As one example, the message 174 may comprise (e.g., if the coaching domain is active transport) the following personalized message: “Hi Adnan! You have chosen to focus on active transportation this week. Your goal is to spend this week at least 36 minutes of active transportation. I will support your efforts.” Depending on the coaching domain, this message may be updated automatically periodically (e.g., every week). Note that the coaching messages are relevant to a user in the coaching domain based on the targeting of certain personal determinants. It is noted that when the user is in the education or maintenance phases, the user may receive messages on a regular basis. In the education phase, users may receive messages that put their (insufficient) performance into perspective, as well as messages that emphasize the need for, and benefits of, physical activity. In the maintenance (feedback) phase, the user may receive positive feedback on his satisfactory behavior. In some embodiments, a user may receive general messages in all phases, for instance regarding his current step or stair counts or containing universal motivational messages.

The activity data 162, 164 may be presented according to respective small panels showing recent step and floor counts, respectively. A script may update the values presented in the activity data 162, 164 when the user visits the website, when an app (e.g., application software 30A of the smartphone 14) is opened, among other times.

The progress bar 166 comprises a graphical representation of progress towards a goal (e.g., a weekly goal of 70,000 steps). The panel in which the progress bar 166 is presented may include a caption to identify what the progress bar 166 represents (e.g., “Progress to weekly step goal”).

The domain data 168-172 presents a dashboard of physical activity for the chosen domain (e.g., steps, floors, time spent on active transport or leisure time sports activities, etc.) for the current interval of time (e.g., for a week of active transport, floors, sports). In one embodiment, only one of the domain data 168, 170, or 172 is presented, the presented one being that chosen for the current monitored interval of time (e.g., week). In some embodiments, all three domain data 168-172 are shown (e.g., preliminarily when no domain has yet to be selected). In one embodiment, the domain data 168-172 presents a record of physical activity (e.g., based on the monitored activity by the wearable device 12, FIG. 2). In one embodiment, the domain data 168-172 presents population/group averages adjacent user data (e.g., in bar charts). Icons or other input mechanisms may be included in the user interface 158 to enable the user the option to view past performance at a user-selected (or in some embodiments, predetermined) interval. In some embodiments, options may be available to view comparative data for the domain data 168-172, including comparisons by group average, age group, social group, etc.

The rank data 167 lists names of a number of users and representations of performance over a defined period (e.g., horizontal bars visualizing, for instance, a number of steps for the last seven (7) days.

Explaining the social comparisons of the domain data 168-172 further, the messaging system may enable comparisons between the user's performance with others. In the depicted embodiment, two types of social comparisons are presented—one for comparing the performance data for the user to population or group data (e.g., anonymously), and another for listing names along with comparisons of performance. One basis for enabling such comparisons is that healthy lifestyles can be maintained and achieved in the presence of a social support network. In one embodiment, the messaging system implements comparisons by ranking the user's performance within a list of other users' performance. The ranking may automatically update every time, for instance, a user visits the website hosted by the messaging system (or via opening up the app at the smartphone 14 or wearable device 12). In one embodiment, physical activity is used as a basis for the comparison, which may be determined by the number of steps taken by an individual over a defined period (e.g., over the last seven (7) days), and/or which may be based on physical activity in the current coaching domain (e.g., floors, minutes of active transportation, minutes of sports, etc.). In one embodiment, the messaging system may show actual friends of the user in the ranking using a social site API. In some embodiments, anonymity may be maintained as desired by the user or as pre-configured.

It should be appreciated, as indicated above, that other and/or additional information may be presented in the user interface 158 in a single display or sequenced through a plurality of displays (e.g., as prompted by the user or automatically scrolled). In some embodiments, less information may be presented in the user interface 158.

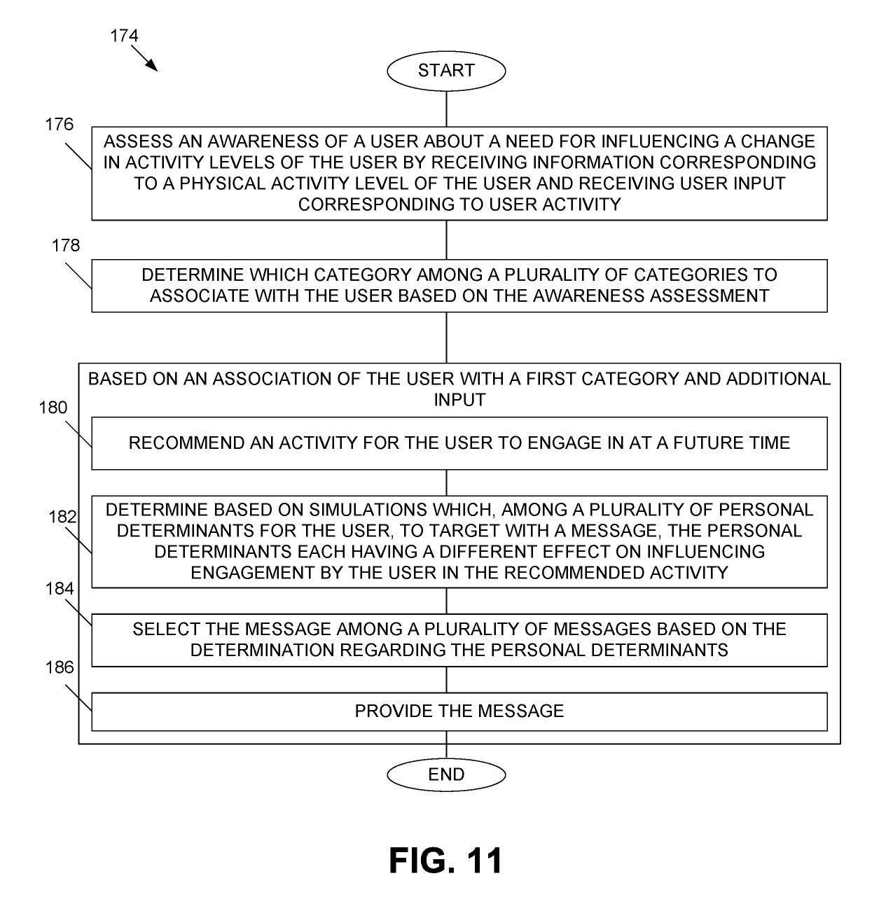

In view of the description above, it should be appreciated that one embodiment of a messaging method (e.g., implemented by the messaging system), depicted in FIG. 11 and referred to as a method 174 and encompassed between start and end designations, comprises assessing an awareness of a user about a need for influencing a change in activity levels of the user by receiving information corresponding to a physical activity level of the user and receiving user input corresponding to user activity (176); determining which category among a plurality of categories to associate with the user based on the awareness assessment (178); based on an association of the user with a first category and additional input: recommending an activity for the user to engage in at a future time (180); determining based on simulations which, among a plurality of personal determinants for the user, to target with a message, the personal determinants each having a different effect on influencing engagement by the user in the recommended activity (182); selecting the message among a plurality of messages based on the determination regarding the personal determinants (184); and providing the message (186).

Note that although four categories are used in the examples above, additional or fewer categories may be used in some embodiments. In some embodiments, additional or fewer domains may be used. Though active transport, stair walking, and sports participation are used as example activity categories for the coaching domain, additional activity categories may be used in some embodiments. For example, some implementations may involve activities that are not necessarily viewed as sports or recreation activities. In one embodiment, a messaging system may be used in the context of sleep coaching for a person with a physical or physiological ailment, such as a person with respiratory issues, including those with sleep apnea, COPD (chronic obstructive pulmonary disease), or asthma. That is, in these circumstances, a medical professional may recommend CPAP (continuous positive air pressure) therapy, which may involve a flow generator, tubing or hose, and mask to provide the positive air pressure from the generator via the hose to the patient. The success of the treatment may be based at least in part on the patient's ability (or tolerance) to continually overcome the positive air pressure (which may cause an unpleasant feeling), or the patient's tolerance to what he or she may perceive as an uncomfortable or constricting mask fit, among other obstacles. In other words, the ability to sleep with the CPAP system is important to success of the therapy. Certain embodiments of a messaging system may be deployed to find the promising coaching determinants and generate messages that help the patient alleviate the discomfort of using the CPAP system during sleep. The patient may input replies to audibly or visually presented questions, repeated over time, via an electronic device (e.g., smartphone, computing equipment associated with the CPAP system, wearable device, etc.). The replies to these questions are used by the reasoning engine in similar manner to that described above. Additional input to the messaging system may include, similar to the above-described embodiments, physiological input from a wearable device, heart rate monitor, etc.

In one embodiment, a claim to an apparatus is disclosed, comprising: a memory comprising executable code; and a processor configured by the executable code to: assess an awareness of a user about a need for influencing a change in activity levels of the user by receiving information corresponding to a physical activity level of the user and receiving user input corresponding to user activity; determine which category among a plurality of categories to associate with the user based on the awareness assessment; based on an association of the user with a first category and additional input: recommend an activity for the user to engage in at a future time; determine based on simulations which, among a plurality of personal determinants for the user, to target with a message, the personal determinants each having a different effect on influencing engagement by the user in the recommended activity; select the message among a plurality of messages based on the determination regarding the personal determinants; and provide the message.

In one embodiment, a claim depending on the preceding claim, wherein the processor is further configured by the executable code to determine which category to associate with the user by evaluating rules that associate an objective binary measure of the physical activity and a subjective binary measure of the user input to each of the plurality of categories.

In one embodiment, a claim depending on any one of the preceding claims, wherein the processor is further configured by the executable code to associate the user with the first category based on either: determining that the objective and subjective binary measures are of equal values; or determining that the objective binary measure is of a first value and the subjective binary measure is of another value.

In one embodiment, a claim depending on any one of the preceding claims, wherein the first category comprises a coaching category.

In one embodiment, a claim depending on any one of the preceding claims, wherein the processor is further configured by the executable code to recommend an activity by recommending one of plural domains, wherein the plural domains include active transport, stair walking, or sports participation.

In one embodiment, a claim depending on any one of the preceding claims, wherein the processor is further configured by the executable code to recommend the activity based on evaluating the information for each of the plural domains.

In one embodiment, a claim depending on any one of the preceding claims, wherein the processor is further configured by the executable code to receive the information from any one or a combination of an activity monitor or via an application running in another device.

In one embodiment, a claim depending on any one of the preceding claims, wherein the processor is further configured by the executable code to perform the evaluation by comparing the information for each domain to a reference value, the reference value determined based on contextual information.

In one embodiment, a claim depending on any one of the preceding claims, wherein the contextual information comprises any one or a combination of location information, active travel options between locations corresponding to the location information, inactive travel options between locations corresponding to the location information, floor numbers at the locations, or availability of stairs at the locations.

In one embodiment, a claim depending on any one of the preceding claims, wherein the processor is further configured by the executable code to receive the contextual information based on user responses to a questionnaire.

In one embodiment, a claim depending on any one of the preceding claims, wherein the processor is further configured by the executable code to recommend one of the plural domains with a lowest evaluation score, the lowest evaluation score corresponding to a largest potential for improvement.

In one embodiment, a claim depending on any one of the preceding claims, wherein the processor is further configured by the executable code to enable the recommendation to be overridden based on user input.

In one embodiment, a claim depending on any one of the preceding claims, wherein the processor is further configured by the executable code to prompt the user to set a goal for the activity, wherein the goal is implemented over a predetermined interval of time.

In one embodiment, a claim depending on any one of the preceding claims, wherein the processor is further configured by the executable code to estimate an effect of improving each personal determinant by running simulations through a computational model of determinants over one or more repeated intervals, wherein the processor is further configured by the executable code to run the simulations by: estimating a current state of each of the personal determinants; inputting values corresponding to the current state; increasing a value for respective one or more targeted personal determinants according to a hypothesized effect of sending a message about the one or more targeted personal determinants; based on the increased value or values, estimating an effect on behavior; and providing an ordered list of personal determinants and a corresponding probability, the ordering based on a most promising effect on the corresponding behavior.

In one embodiment, a claim depending on any one of the preceding claims, wherein the estimating of a current state is based on the additional input comprising user responses to questions presented after the activity is recommended.

In one embodiment, a claim depending on any one of the preceding claims, wherein the processor is further configured by the executable code to select the messages based on the determined personal determinant and by filtering out messages from the plurality of messages based on a lack of correspondence between the filtered out messages and the first category and the recommended activity and further based on any one or a combination of day and time, an occupational status of the user, user responses to questions, determined progress of the user towards a goal, and weather information.

In one embodiment, a claim depending on any one of the preceding claims, wherein the processor is further configured by the executable code to personalize the selected message by populating fields of the selected message with user-specific data.

In one embodiment, a claim depending on any one of the preceding claims, wherein the processor is further configured by the executable code to determine which category to associate by considering categories that include a feedback category and an education category.

In one embodiment, a claim to a computer-implemented method is disclosed, comprising: assessing an awareness of a user about a need for influencing a change in activity levels of the user by receiving information corresponding to a physical activity level of the user and receiving user input corresponding to user activity; determining which category among a plurality of categories to associate with the user based on the awareness assessment; based on an association of the user with a first category and additional input: recommending an activity for the user to engage in at a future time; determining based on simulations which, among a plurality of personal determinants for the user, to target with a message, the personal determinants each having a different effect on influencing engagement by the user in the recommended activity; selecting the message among a plurality of messages based on the determination regarding the personal determinants; and providing the message.

In one embodiment, a claim to a non-transitory computer readable medium is disclosed, the non-transitory computer readable medium encoded with instructions executable by a processor or processors that causes the processor or processors to: assess an awareness of a user about a need for influencing a change in activity levels of the user by receiving information corresponding to a physical activity level of the user and receiving user input corresponding to user activity; determine which category among a plurality of categories to associate with the user based on the awareness assessment; based on an association of the user with a first category and additional input: recommend an activity for the user to engage in at a future time; determine based on simulations which, among a plurality of personal determinants for the user, to target with a message, the personal determinants each having a different effect on influencing engagement by the user in the recommended activity; select the message among a plurality of messages based on the determination regarding the personal determinants; and provide the message.

Note that various combinations of the disclosed embodiments may be used, and hence reference to an embodiment or one embodiment is not meant to exclude features from that embodiment from use with features from other embodiments. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. A computer program may be stored/distributed on a suitable medium, such as an optical medium or solid-state medium supplied together with or as part of other hardware, but may also be distributed in other forms. Any reference signs in the claims should be not construed as limiting the scope.

At least the following is claimed:

1. An apparatus, comprising:
a memory comprising executable code; and
a processor configured by the executable code to:
assess an awareness of a user about a need for influencing a change in activity levels of the user by receiving information corresponding to a physical activity level of the user and receiving user input corresponding to user activity;
determine which category among a plurality of categories to associate with the user based on the awareness assessment;
based on an association of the user with a first category and additional input:
recommend an activity for the user to engage in at a future time;
determine based on simulations which, among a plurality of personal determinants for the user, to target with a message, the personal determinants each having a different effect on influencing engagement by the user in the recommended activity;
select the message among a plurality of messages based on the determination regarding the personal determinants; and
provide the message.

2. The apparatus of claim 1, wherein the processor is further configured by the executable code to determine which category to associate with the user by evaluating rules that associate an objective binary measure of the physical activity and a subjective binary measure of the user input to each of the plurality of categories.

3. The apparatus of claim 2, wherein the processor is further configured by the executable code to associate the user with the first category based on either:
determining that the objective and subjective binary measures are of equal values; or
determining that the objective binary measure is of a first value and the subjective binary measure is of another value.

4. The apparatus of claim 1, wherein the first category comprises a coaching category.

5. The apparatus of claim 1, wherein the processor is further configured by the executable code to recommend an activity by recommending one of plural domains, wherein the plural domains include active transport, stair walking, or sports participation.

6. The apparatus of claim 5, wherein the processor is further configured by the executable code to recommend the activity based on evaluating the information for each of the plural domains.

7. The apparatus of claim 6, wherein the processor is further configured by the executable code to receive the information from any one or a combination of an activity monitor or via an application running in another device.

8. The apparatus of claim 6, wherein the processor is further configured by the executable code to perform the evaluation by comparing the information for each domain to a reference value, the reference value determined based on contextual information.

9. The apparatus of claim 8, wherein the contextual information comprises any one or a combination of location information, active travel options between locations corresponding to the location information, inactive travel options between locations corresponding to the location information, floor numbers at the locations, or availability of stairs at the locations.

10. The apparatus of claim 8, wherein the processor is further configured by the executable code to receive the contextual information based on user responses to a questionnaire.

11. The apparatus of claim 8, wherein the processor is further configured by the executable code to recommend one of the plural domains with a lowest evaluation score, the lowest evaluation score corresponding to a largest potential for improvement.

12. The apparatus of claim 11, wherein the processor is further configured by the executable code to enable the recommendation to be overridden based on user input.

13. The apparatus of claim 1, wherein the processor is further configured by the executable code to prompt the user to set a goal for the activity, wherein the goal is implemented over a predetermined interval of time.

14. The apparatus of claim 1, wherein the processor is further configured by the executable code to estimate an effect of improving each personal determinant by running simulations through a computational model of determinants over one or more repeated intervals, wherein the processor is further configured by the executable code to run the simulations by:
estimating a current state of each of the personal determinants;
inputting values corresponding to the current state;
increasing a value for respective one or more targeted personal determinants according to a hypothesized effect of sending a message about the one or more targeted personal determinants;
based on the increased value or values, estimating an effect on behavior; and
providing an ordered list of personal determinants and a corresponding probability, the ordering based on a most promising effect on the corresponding behavior.

15. The apparatus of claim 14, wherein the estimating of a current state is based on the additional input comprising user responses to questions presented after the activity is recommended.

16. The apparatus of claim 14, wherein the processor is further configured by the executable code to select the messages based on the determined personal determinant and by filtering out messages from the plurality of messages based on a lack of correspondence between the filtered out messages and the first category and the recommended activity and further based on any one or a combination of day and time, an occupational status of the user, user responses to questions, determined progress of the user towards a goal, and weather information.

17. The apparatus of claim 16, wherein the processor is further configured by the executable code to personalize the selected message by populating fields of the selected message with user-specific data.

18. The apparatus of claim 16, wherein the processor is further configured by the executable code to determine which category to associate by considering categories that include a feedback category and an education category.

19. A computer-implemented method, comprising:
assessing an awareness of a user about a need for influencing a change in activity levels of the user by receiving information corresponding to a physical activity level of the user and receiving user input corresponding to user activity;
determining which category among a plurality of categories to associate with the user based on the awareness assessment;
based on an association of the user with a first category and additional input:
recommending an activity for the user to engage in at a future time;
determining based on simulations which, among a plurality of personal determinants for the user, to target with a message, the personal determinants each having a different effect on influencing engagement by the user in the recommended activity;
selecting the message among a plurality of messages based on the determination regarding the personal determinants; and
providing the message.

20. A non-transitory computer readable medium encoded with instructions executable by a processor or processors that causes the processor or processors to:
assess an awareness of a user about a need for influencing a change in activity levels of the user by receiving information corresponding to a physical activity level of the user and receiving user input corresponding to user activity;
determine which category among a plurality of categories to associate with the user based on the awareness assessment;
based on an association of the user with a first category and additional input:
recommend an activity for the user to engage in at a future time;
determine based on simulations which, among a plurality of personal determinants for the user, to target with a message, the personal determinants each having a different effect on influencing engagement by the user in the recommended activity;
select the message among a plurality of messages based on the determination regarding the personal determinants; and
provide the message.
